SEQUENCE LISTING (10 sequences)

SEQ ID NO: 1 - DNA, 20 bp, artificial
  forward primer for hTGM2
agggtgacaagagcgagatg

SEQ ID NO: 2 - DNA, 19 bp, artificial
  reverse primer for hTGM2
tggtcattcacgactccac

SEQ ID NO: 3 - DNA, 19 bp, artificial
  forward primer for mTGM1
gcccttgagctcctcattg

SEQ ID NO: 4 - DNA, 20 bp, artificial
  reverse primer for mTGM1
cccttacccactgggatgat

SEQ ID NO: 5 - DNA, 20 bp, artificial
  forward primer for mTGM2
ggtgatcctcgcttgagtgt

SEQ ID NO: 6 - DNA, 21 bp, artificial
  reverse primer for mTGM2
ctccaaatcacacctctccag

SEQ ID NO: 7 - DNA, 20 bp, artificial
  forward primer for mTGM3
gatcacagctgtttgcaagg

SEQ ID NO: 8 - DNA, 19 bp, artificial
  reverse primer for mTGM3
catgagcctgttccagcac

SEQ ID NO: 9 - DNA, 19 bp, artificial
  forward primer for mTGM5
tccatccagctgtctgtgg

SEQ ID NO: 10 - DNA, 20 bp, artificial
  reverse primer for mTGM5
agggccacctctagtcctgt

(19) United States
(12) Patent Application Publication
(10) Pub. No.: US 2014/0050779 A1
(43) Pub. Date: Feb. 20, 2014

(54) TRANSGLUTAMINASE-2 INHIBITORS AND USES THEREOF

(71) Applicants: Roger W. Beuerman, Singapore (SG); Veluchamy Amutha Barathi, Singapore (SG); Louis Tong, Singapore (SG)

(72) Inventors: Roger W. Beuerman, Singapore (SG); Veluchamy Amutha Barathi, Singapore (SG); Louis Tong, Singapore (SG)

(73) Assignee: SINGAPORE HEALTH SERVICES PTE. LTD., Singapore (SG)

(21) Appl. No.: 13/984,508

(22) PCT Filed: Feb. 8, 2012
(86) PCT No.: PCT/SG2012/000037

§ 371 Date: Oct. 28, 2013

Related U.S. Application Data
(60) Provisional application No. 61/440,886, filed on Feb. 9, 2011.

(51) Int. Cl. A61K 31/407; A61K 31/7105; C12Q 1/68; A61K 31/45; G01N 33/50; A61K 31/4355; A61K 31/4025

(52) U.S. Cl. CPC A61K 31/407; A61K 31/4355; A61K 31/7105; A61K 31/4025; A61K 31/45; G01N 33/5023; C12Q 1/6883
USPC 424/450; 514/291; 514/411; 514/44 A; 514/422; 514/327; 435/6.13; 435/6.11; 435/6.12; 506/9

(57) ABSTRACT

The present invention refers to a method of treating a disease or disorder associated with the expression of at least one transgluaminase-2 and a method of identifying a candidate transglutaminase-2 inhibitor.

### CROSS-REFERENCE TO RELATED APPLICATION

This application makes reference to and claims the benefit of priority of an application for “Transglutaminase-2 inhibitors and uses thereof” filed on Feb. 9, 2011 with the United States Patent and Trademark Office, and there duly assigned application No. 61/440,886. The content of said application filed on Feb. 9, 2011 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to methods of treating a disease or disorder associated with the expression of transglutaminase-2 as well as methods of identifying a candidate transglutaminase-2 inhibitor.

### BACKGROUND OF THE INVENTION

Myopia is the most common refractive disorder worldwide, reaching epidemic proportions in particular in many Asian countries including Singapore, where the prevalence is 80% by 18 years of age (Saw S M., 2003, A synopsis of the prevalence rates and environmental risk factors for myopia. Clin Exp Optom. 86(5):289-94. Review). Myopia incurs significant socio-economic cost and the annual direct cost of myopia for an affected individual was estimated to be S$125 (USD 95) (Lim, M. C., Gazzard, G., Sim, E. L., Tong, L., Saw, S. M., 2009. Direct costs of myopia in Singapore. Eye (Lond) 23, 1086-1089). This annual cost is likely to increase due to the increasing number of children developing high myopia greater than 6D. As a consequence of high myopia (myopia greater than 6D), there is an increasing risk of developing blinding retinal pathology later in life. Refractive surgeries are only aimed at reducing the need of relying on visual aids, rather than providing a cure for myopia. Due to the above reasons, there is a need to develop an effective treatment to prevent or treat myopia.

Myopia results from axial elongation of the posterior segment of the eye resulting in image formation in front of the retina. The fibroblasts of the sclera, the outer tunic of the eye regulate the growth of the sclera in myopia (Gilmartin B. Myopia: precedents for research in the twenty-first century. Clin Experiment Ophthalmol. 32, 305, 2004). Hence, modulation of connective tissue molecules in the sclera, for example the fibroblasts of the sclera may represent a strategy for arresting myopia development, regardless of the initiating stimulus.

To date, there are several interventions that can decrease myopia progression. Atropine, a non-selective muscarinic antagonist, has been widely used to treat myopia and is used in children (Barathi, V. A., Beuerman, R. W., Schaeffel, F., 2009a. Effects of unilateral topical atropine on binocular pupil responses and eye growth in mice. Vision Res 49, 383-387; Chua, W. H., Balakrishnan, V., Chan, Y. H., Tong, L., Ling, Y., Quah, B. L., Tan, D., 2006. Atropine for the treatment of childhood myopia. Ophthalmology 113, 2285-2291). Even though muscarinic receptor subtypes may be involved in scleral remodeling through their action on the scleral fibroblasts, the mode of action and the mechanism is unknown. A complication of this data is that there are muscarinic receptors on many different tissues in the eye. To date, muscarinic acetylcholine receptors (mAChRs), the binding sites for muscarinic agents, are known to be involved in myopia. mAChRs, which are made up of seven transmembrane domains, belong to a class of metabotropic receptors which elicit downstream responses via heterotrimer G proteins (GTP). Numerous studies had characterized the muscarinic receptors into five different subtypes; M₁-M₅. Pirenzepine, a muscarinic receptor M₁-specific antagonist has also shown to inhibit myopia progression in mammalian and avian models.

In this context, many different experimental animal models (chick, rabbit, tree shrew, macaque and etc) have been used for studies of emmetropization, the progression of the development of normal vision as well as myopia generation. These animal models were used to characterize the optical parameters of and study the mechanisms of induced myopia. Studies of the chick eye have formed the basis for several hypotheses of myopic development, but the chick does not possess a mAChR1 or a foveal or retinal blood supply. It is unclear whether these differences alter the pathways of emmetropization. Moreover, the sclera structure is unlike that found in mammals. In this regard, a mouse myopia model has been recently developed (Schaeffel F, et. al., (2004) Measurement of refractive state and deprivation myopia in two strains of mice (Optom Vis Sci. 81(2):99-110; Faulkner A E, et. al., (2007) Head-mounted goggles for murine form deprivation myopia. J Neurosci Methods. 2007 Mar. 30; 161(1): 96-100). Mouse myopia model was developed because of the availability of the whole genome sequence, comprehensive protein database and more importantly, the availability of molecular tools like whole genome gene-chip. With these mouse models, as well as non-invasive method for measuring and monitoring axial length, it is possible to monitor the progress of myopia in the same individual without the need to sacrifice animal. Although animal models namely tree shrew, chick and primates were used for atropine studies, studies on mAChR antagonists' treatment in mice models of myopia have not been performed to date.

TG-2 or tissue transglutaminase is a member of the transglutaminase superfamily. They are widely expressed throughout the body and are involved in many cellular processes such as wound healing, apoptosis and cell migration. TG-2 was predominantly found in ocular tissues and was reported to be highly expressed in cultured human retinal pigment epithelial (RPE) cells (Priglinger, S. G., et. al., 2003, Tissue transglutaminase as a modifying enzyme of the extracellular matrix in PVR membranes. Invest Ophthalmol Vis Sci 44, 355-364). In the RPE cells, transglutaminase activity was demonstrated to be regulated by intracellular calcium and GTP. Their functions greatly depend on their localization. TG-2 has been found to have GTPase activity and intracellular G protein signalling via the α_{1B}/α_{1D} adrenergic receptors, function as protein kinase, protein disulfide isomerase and adaptor protein (Iismaa S. E. et al, Physiol. Rev., 89: 991-1023, 2009). However, the pathway(s) and molecular mechanisms by which TG2 is externalized are largely unknown.

Therefore, there remains a need to develop an effective treatment to prevent myopia.

### SUMMARY OF THE INVENTION

In one aspect the invention provides a method of treating a disease or disorder associated with the expression of transglutaminase-2 (TGM-2). The method includes administering to a subject a TG-2 inhibitor. The TG-2 inhibitor is selected from the group consisting of darifenacin or an analogue thereof; 1,1-Dimethyl-4-diphenylacetoxypiperidinium iodide (4-DAMP) or an analogue thereof; a nucleic acid molecule that inhibits expression of TGM-2; and himbacine or an analogue thereof. The analogue of himbacine may be a compound of Formula I:

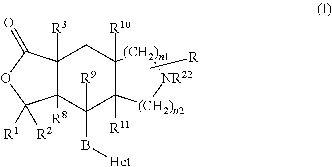

or a pharmaceutically acceptable salt thereof, wherein:

R is 1 to 3 substituents independently selected from the group consisting of H, C₁-C₆ alkyl, halogen, hydroxy, amino, (C₁-C₆)alkyl-amino, (C₁-C₆)-dialkylamino, (C₁-C₆)alkoxy, —COR¹⁶, —COOR¹⁷, —SOR¹⁶, —SO₂R¹⁶, —SO₂NR¹⁷R¹⁸, —NR¹⁷SO₂R₁₈, NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, C₃-C₆cycloalkyl, aryl(C₁-C₆)alkyl, hydroxy(C₁-C₆)alkyl, amino-(C₁-C₆)-alkyl, aryl and thio(C₁-C₆)alkyl;

R¹ and R² are independently selected from the group consisting of H, C₁-C₆ alkyl, fluoro(C₁-C₆)alkyl, difluoro(C₁-C₆)alkyl, trifluoro-(C₁-C₆)alkyl, C₃-C₆cycloalkyl, C₂-C₆ alkenyl, aryl(C₁-C₆)alkyl, hydroxy-(C₁-C₆)alkyl, amino(C₁-C₆)alkyl, aryl and thio(C₁-C₆)alkyl; or and R¹ and R² together form an ═O group;

R³ is H, hydroxy, C₁-C₆alkoxy, aryloxy, aryl(C₁-C₆)alkyloxy, heteroaryloxy, heteroaryl(C₁-C₆)alkyloxy, (C₃-C₆)cycloalkyloxy, —SOR¹⁶, —SO₂R¹⁷, —SO₂NR¹⁸R¹⁹, —SR¹⁸, —SO₃H, —C(O)OR¹⁷, —C(O)NR¹⁸R¹⁹, —OC(O)R³², —OC(O)NR³³R³⁴, —(CR³³R³⁴)ₙOR³², —NR⁴R⁵, —NR³³COOR³², —NR³³COR³², —NR³³S(O)₂R³², —NR³³CONR³³R³⁴, —NR³³S(O)₂NR³³R³⁴, —(CR³³R³⁴)ₙNR⁴R⁵, —(CR³³R³⁴)ₙNR³³COR³², —(CR³³R³⁴)ₙNR³³COR³², —(CR³³R³⁴)ₙNR³³S(O)₂R³², —(CR³³R³⁴)ₙNR³³CONR³³R³⁴, —(CR³³R³⁴)ₙNR³³S(O)₂NR³³R³⁴, (C₁-C₆)alkyl, halogen, C₃-C₆cycloalkyl, C₂-C₆alkenyl, —CN, aryl, heteroaryl, heterocycloalkyl, —P(O)(OR⁷)₂ or (C₁-C₆)alkyl substituted by 1 to 3 substituents independently selected from the group consisting of halogen, —OH, —NH₂, aryl, —COOH, —SO₃H, thio and (C₁-C₆)alkylthio;

n is 1, 2, 3 or 4;

n1 and n2 are independently 0-3, provided both are not 0;

Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of C₁-C₆alkyl; —NR⁴R⁵; —NHCOR²⁶; —NHSO₂R¹⁶; R²¹-aryl; aryl wherein adjacent carbons form a ring with a methylenedioxy group; and R²¹-heteroaryl;

R⁴ and R⁵ are independently selected from the group consisting of H, C₁-C₆ alkyl, phenyl, benzyl and C₃-C₆ cycloalkyl, or R⁴ and R⁵ together are —(CH₂)₃—, —(CH₂)₄—, —(CH₂)₅— or —(CH₂)₂NR⁷—(CH₂)₂— and form a ring with the nitrogen to which they are attached;

R⁷ is H or (C₁-C₆)alkyl;

R⁸, R¹⁰ and R¹¹ are independently selected from the group consisting of R¹ and —OR¹;

R⁹ is H, OH, —NR⁴R⁵, C₁-C₆alkoxy, halogen or halo(C₁-C₆)alkyl;

B is —(CH₂)n₃- or cis or trans —(CH₂)n₄CR¹²═CR^{12a}(CH₂)n₅, wherein n₃ is 0-5, n₄ and n₅ are independently 0-2, and R¹² and R^{12a} are independently selected from the group consisting of H, C₁-C₆ alkyl and halogen;

R¹⁶ and R^{16a} are independently selected from the group consisting of C₁-C₆ alkyl, phenyl and benzyl;

R¹⁷, R¹⁸, and R¹⁹ are independently selected from the group consisting of H, C1-C₆alkyl, phenyl and benzyl;

R²¹ is 1 to 3 substituents independently selected from the group consisting of H, —CF₃, —OCF₃, halogen, —NO₂, —CN, C₁-C₆alkyl, C₁-C₆alkoxy, —NH₂, (C₁-C₆)-alkyl-amino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —CONR²⁴R²⁵, —NHCOR¹⁶, —NHSO₂R¹⁶, —NHSO₂CH₂CF₃, —SO₂NR²⁴R²⁵, —NR²⁹C(O)NR²⁴R²⁵, —SO₂R³⁰, —P(O)(OR²⁹)₂, aryl, aryl(C₁-C₆)alkyl, heteroaryl, heterocycloalkyl, and —CR²⁹(═NOR²⁸);

R²² is —COR²³, —S(O)R³¹, —S(O)₂R³¹, —SO₂NR²⁴R²⁵ or —COOR²⁷;

R²³ is halo (C₁-C₆)alkyl; C₂-C₆alkenyl; halo (C₂-C₆)alkenyl; C₂-C₆alkynyl; C₃-C₇-cycloalkyl; (C₃-C₇)cycloalkyl(C₁-C₆)alkyl; (C₃-C₇)cycloalkyl substituted by 1 to 3 substituents selected from the group consisting of halo, (C₁-C₃)alkoxy(C₁-C₃)alkyl, hydroxy and C₁-C₆alkoxy; aryl; aryl(C₂-C₆)alkyl; heteroaryl; heterocycloalkyl; (C₁-C₆)alkyl substituted by 1-3 substituents independently selected from —COOH and —SO₃H; or

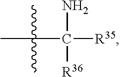

wherein R³⁵ and R³⁶ are independently selected from the group consisting of H, alkyl, or R³⁷-substituted C₁-C₆alkyl, wherein R³⁷ is selected from the group consisting of HO—, HS—, CH₂S—, —NH₂, phenyl, p-hydroxyphenyl and indolyl;

R²⁴ and R²⁵ are independently selected form the group consisting of H, C₁-C₆alkyl, halo(C₁-C₆)alkyl, C₂-C₆alkenyl, halo(C₂-C₆)alkyl, C₂-C₆alkynyl, aryl, aryl-(C₁-C₆)alkyl, C₃-C₇-cycloalkyl, halo(C₃-C₇)cycloalkyl, (C₁-C₃)alkoxy(C₁-C₃)-alkyl, hydroxy and C₁-C₆alkoxy;

R²⁶ is C₃-C₇-cycloalkyl, aryl, aryl-(C₁-C₆)alkyl, heteroaryl, heteroaryl-(C₁-C₆)alkyl or (C₁-C₆)alkylamino;

R²⁷ is C₁-C₆alkyl, phenyl, benzyl, (C₁-C₃)alkoxy(C₁-C₃)-alkyl, (C₃-C₇)-cycloalkyl, carboxy(C₁-C₆)alkyl, sulfo(C₁-C₆)alkyl, or (C₁-C₆)alkyl substituted by NR¹⁸R¹⁹ and carboxy;

R²⁸ is H, C₁-C₆alkyl, phenyl, benzyl or (C₁-C₆)alkoxy(C₁-C₃)alkyl;

R²⁹ and R³⁰ are independently selected from the group consisting of H and C₁-C₆alkyl;

R³¹ is (C₁-C₆)alkyl; halo(C₁-C₆)alkyl; C₂-C₆alkenyl; halo(C₂-C₆)alkyl; C₂-C₆alkynyl; C₃-C₇-cycloalkyl; (C₃-C₇)cycloalkyl substituted by 1 to 3 substituents selected from the group consisting of halo, (C₁-C₃)alkoxy(C₁-C₃)alkyl, hydroxy and C₁-C₆alkoxy; aryl; aryl (C₁-C₆)alkyl; heteroaryl; heterocycloalkyl; (C₁-C₆)alkyl substituted by 1-3 substituents independently selected from —COOH and —SO₃H; or (C₁-C₆)alkoxy;

R³² is R³⁵—(C₁-C₆)alkyl, R³⁵—(C₃-C₇)cycloalkyl, R³⁵—(C₂-C₆)alkenyl, R³⁵—(C₂-C₆)-alkynyl or R³⁵-aryl, wherein R³⁵ is 1 or 2 substituents independently selected from the group consisting of H, —COOH, —NH₂, —SO₃H, ═O and ═NOR²⁸; and

R³³ and R³⁴ are independently selected from the group consisting of H, (C₁-C₆)alkyl and C₃-C₇-cycloalkyl.

In another aspect the invention provides a method of identifying a candidate TG-2 inhibitor. The method includes a) contacting a TGM-2 expressing cell with a solution containing a putative TG-2 inhibitor to be identified. The method further includes measuring whether the expression of TGM-2 is inhibited in the cell.

### BRIEF DESCRIPTION OF THE DRAWINGS

In the following description, various embodiments of the invention are described with reference to the following drawings, in which:

FIG. 1A shows the presence of transglutaminases (TGs) in the mouse eye by immunostaining. To determine the presence of TGs in the mouse eye, the whole eye sections (5 microns) were stained using anti mouse TG-1 (“TGase-1”), TG-2 (“TGase-2”), TG-3 (“TGase-3”) and TG-5 (“TGase-5) rabbit IgG-fluorescein conjugates. The negative control section was incubated with 2% goat serum without the respective primary antibodies. Positive immunostaining of TGs were identified in the mouse cornea, retina pigment epithelium (RPE), choroid and sclera, as compared to the negative control. The localization of TG-2 in cornea is different from the other 3 TGs. TG-1, TG-3, TG-5 were localized in the entire mouse corneal epithelium, stroma and endothelium but TG-2 was present only in the corneal subepithelium (“CS”) and stroma (“S”) (see white arrows). Error bar=50 μM. DAPI stains nuclei (indicated by the white circles and the white boundaries) and FITC stains cell membrane and cytoplasm. Magnification at 200×.

FIG. 1B shows the localization of TG-1 (“TGase-1”), TG-2 (“TGase-2”), TG-3 (“TGase-3”) and TG-5 (“TGase-5”) in the human scleral tissue with scleral staining between the collagen fibre bundles by immunofluorescent staining. No immunostaining was found in the negative control. Error bar=50 μM. Arrow indicates the scleral fibroblasts. DAPI stains nuclei (indicated by the white circles) and FITC stains cell membrane and cytoplasm. Magnification at 200×.

FIG. 2A shows the localization of TG-1 (“TGase-1”), TG-2 (“TGase-2”), TG-3 (“TGase-3”) in the mouse palpebral (P), formiceal (F) and bulbar (B) conjunctiva but not TG-5 (“TGase-5”), by immunofluorescent staining. Error bar=50 μM. DAPI stains nuclei (indicated by white circles) and FITC stains cell membrane and cytoplasm. Magnification at 200×.

FIG. 2B shows the localization of TGs in mouse meibomian glands. All TGs were expressed in mouse meibomian glands but TG-2 was weakly detected. Error bar=50 μM. Arrow indicates the meibomian gland. DAPI stains nuclei (indicated by white circles) and FITC stains cell membrane and cytoplasm. Magnification at 200×.

FIG. 3A shows that TG-1, TG-2, TG-3 and TG-5 are expressed by human scleral fibroblasts. The cultured scleral fibroblasts expressed all 4 TGs at cellular level. TG-1, TG-3 and TG-5 were located in the cytosolic and membrane compartments only whereas TG-2 was present in cell nucleus along with cytosolic and membrane compartment. Error bar=50 μM. Magnification at 200×.

FIG. 3B shows that TG-1, TG-2, TG-3 and TG-5 are expressed by mouse scleral fibroblasts. The cultured scleral fibroblasts expressed all 4 TGs at cellular level. TG-1, TG-3 and TG-5 were located in the cytosolic and membrane compartments only whereas TG-2 was present in cell nucleus along with cytosolic and membrane compartment. Error bar=50 μM. Magnification at 200×.

FIG. 4 shows a Western Blot image detecting TG-1, TG-2, TG-3 and TG-5 proteins in mouse and human scleral fibroblasts. β-tubulin was used as a loading control. TG-1: 90 kDa, TG-2: 82 kDa, TG-3: 77 kDa, TG-5: 80 kDa and β-tubulin (loading control): 55 kDa.

FIG. 5A shows a Western Blot image detecting TG proteins in human scleral fibroblasts after atropine treatment. P2 cultured human scleral fibroblasts were treated with atropine at concentrations of 0.01 μM, 0.1 μM, 1 μM, 5 μM and 10 μM for 5 days. Following 5 days of treatment, the total cellular protein was extracted from these cells and TG proteins detected via Western Blot analysis. It can be observed that the TG-1, 2 and 5 protein levels were reduced after atropine treatment. However, TG-3 protein level was increased after receiving atropine. β-tubulin was used as a loading control.

FIG. 5B shows a Western Blot image detecting TG proteins in mouse scleral fibroblasts after atropine treatment. P2 cultured mouse scleral fibroblasts were treated with atropine at concentrations of 0.01 μM, 0.1 μM, 1 μM, 5 μM and 10 μM for 5 days. Following 5 days of treatment, the total cellular protein was extracted from these cells and TG proteins detected via Western blot analysis. It can be observed that the TG-1, 2 and 5 protein levels were reduced after atropine treatment. However, TG-3 protein level was increased after receiving atropine. β-tubulin was used as a loading control.

FIG. 6A shows a Western Blot image detecting TG proteins in human scleral fibroblasts after carbachol treatment. P2 cultured human scleral fibroblasts were treated with carbachol at concentrations of 0.01 μM, 0.1 μM, 1 μM, 5 μM and 10 μM for 5 days. Following 5 days of treatment, the total cellular protein was extracted from these cells and TG proteins detected via Western blot analysis. It can be observed that the TG-1, TG-2 and TG-5 protein levels were increased after carbachol treatment. However, TG-3 protein level was decreased after receiving carbachol. β-tubulin was used as a loading control.

FIG. 6B shows a Western Blot image detecting TG proteins in mouse scleral fibroblasts after carbachol treatment. P2 cultured mouse scleral fibroblasts were treated with carbachol at concentrations of 0.01 μM, 0.1 μM, 1 μM, 5 μM and 10 μM for 5 days. Following 5 days of treatment, the total cellular protein was extracted from these cells and TG proteins detected via Western blot analysis. It can be observed that the TG-1, 2 and 5 protein levels were increased after carbachol treatment. However, TG-3 protein level was decreased after receiving carbachol. β-tubulin was used as a loading control.

FIG. 7 shows a bar chart illustrating the results of TGases mRNA expression levels of mouse scleral fibroblasts following with stimulation of muscarinic agents (i.e. atropine or carbachol). P2 cultured mouse scleral fibroblasts were treated with atropine or carbachol at different concentrations of 0.01 μM, 0.1 μM, 1 M, 5 μM and 10 μM for 5 days. Following 5 days of treatment, the total RNA was extracted from these cells and TGases transcript level was quantified via qPCR analysis. Height of bars show the means of three independent samples and error bars represent standard deviation. Values are normalized against GAPDH house keeping genes. The TGases 1, 2 and 5 transcript levels were down regulated after receiving atropine (A, B and D respectively) at all concentrations. However, TGase-3 transcript was upregulated in the atropine treated cells (C). The opposite findings were observed with carbachol treatment in both TGase 2 (E) and TGase 3 (F) at all concentrations. However, TGases 1 and 5 were increased only by relatively higher concentrations of carbachol (G and H respectively).

FIG. 8 shows the cell proliferation growth of scleral fibroblasts (SF) derived from TG-2 mutant mice and wild type (WT) SF for 7 hours in an SF cell proliferation assay.

FIG. 9A shows the immunohistochemistry staining of the mouse eye sections for wild type mice (indicated as M₁^{+/+}, M₂^{+/+}, M₃^{+/+}, M₄^{+/+}, M₅^{+/+}), heterozygous M₁-M₅-silenced mice (indicated as M₁^{+/-}, M₂^{+/-}, M₃^{+/-}, M₄^{+/-}, M₅^{+/-}) and muscarinic M₁-M₅ receptors knockout mice (indicated as M₁^{-/-}, M₂^{-/-}, M₃^{-/-}, M₄^{-/-}, M₅^{-/-}) using antibodies specific for the respective muscarinic receptors M₁ to M₅. Immuno-staining of wild type mice (M₁^{+/+}, M₂^{+/+}, M₃^{+/+}, M₄^{+/+}, M₅^{+/+}) showed clear staining for the presence of all five muscarinic receptors M₁ to M₅ in the sclera, RPE and retinal of the mouse. Mouse eye incubated with 2% goat serum and labeled with FITC secondary antibody (control). In contrast to the wild type staining, the knockout mouse for muscarinic receptors M₁-M₅ (M₁^{-/-}, M₂^{-/-}, M₃^{-/-}, M₄^{-/-}, M₅^{-/-}) showed weak staining for the antibodies reflecting the absence of the muscarinic receptors in the knockout mouse.

FIG. 9B shows a Western Blot image that detects the presence or absence of M₁ to M₅ receptors in the respective M₁^{+/+} to M₅^{+/+} wild type mice, heterozygous M₁^{+/-} to M₅^{+/-}-silenced mice and muscarinic M₁^{-/-} to M₅^{-/-} receptors knockout mice. The immunoreactive bands corresponding to each muscarinic receptor (M₁ to M₅) from wild type mouse scleral tissue confirms the presence of M₁-M₅ receptors. The absence of immunoreactive bands corresponding to each muscarinic receptor from the respective muscarinic M₁^{-/-} to M₅^{-/-} knockout mice sclera tissue confirms the absence of M₁ to M₅ receptors in the respective knock-out mice sclera.

FIG. 10A shows a plot illustrating the results of the transamidase activity of TG-2 in 100 μg of protein lysate from human scleral fibroblasts. Values were normalized against control values. The human scleral fibroblasts were treated with atropine, pirenzepine, AFDX-116, himbacine, 4-DAMP, darifenacin and tropicamide at baseline, 0.1 μM, 1 μM, 10 μM and 100 μM for 5 days. The transamidase activity of endogenous cellular TG-2 activity was reduced by antagonists' treatment in a concentration-dependent manner in human scleral fibroblasts. TG-2 activity was most significantly reduced with himbacine treatment and also with darifenacin at all concentrations. In particular, when treated with himbacine (0.1 μM, 1 μM, 10 μM & 100 μM) and darifenacin (0.1 μM, 1 μM & 10 μM), the TG-2 activity was more significantly reduced as compared to atropine. The values represented the means of three independent samples and error bars represent standard deviation, n=3, *p<0.05, **p<0.01.

FIG. 10B shows a plot illustrating the results of the transamidase activity of TG-2 in 100 μg of protein lysate from mouse cultured scleral fibroblasts. Values were normalized against control values. The mouse scleral fibroblasts were treated with atropine, pirenzepine, AFDX-116, himbacine, 4-DAMP, darifenacin and tropicamide at baseline, 0.1, 1, 10 and 100 μM for 5 days. The transamidase activity of endogenous cellular TG-2 activity was reduced by antagonists' treatment in a concentration-dependent manner in mouse scleral fibroblasts. Moreover TG-2 activity was most significantly reduced by atropine at 0.1 μM and this was quite saturated at 1 μM, 10 μM and 100 μM. TG2 activity was dose dependent with himbacine however TG2 activity was most significantly reduced with high concentration (100 μM) as compared to atropine. The values represented the means of three independent samples and error bars represent standard deviation, n=3, *p<0.05, **p<0.01.

FIG. 11A shows the in vivo measurements of axial length of myopic muscarinic receptor knockout mice. M1^{-/-}, M4^{-/-} and M5^{-/-} muscarinic receptor knockout mice axial growth was significantly 250 μm longer than the M2^{-/-} mouse and 300 μm longer than the M3^{-/-} mouse (indicated as unshaded bars). The wild type muscarinic receptor mice M₁^{+/+}, M₂^{+/+}, M₃^{+/+}, M₄^{+/+} and M₅^{+/+} (indicated as shaded bars) mice axial growth was not significantly different. Error bars=SD. ANOVA T test p<0.05.

FIG. 11B shows the measurements of refractive state of muscarinic receptor knockout mice. The M₂ and M₃ knock-out mice mediate the myopia formation in mice. Error bars=SD. ANOVA T test p<0.05.

FIG. 12A shows the expression of Transglutaminase-2 in eight weeks old murine tissue and cells. Immunofluorescent staining images using primary antibody against TG-2 in murine sclera. Top: Control eye. Arrow indicates scleral fibroblast. Scale bar=50 μm. Inset: enlarged image of fibroblast. Bottom: Lens-induced myopic eye.

FIG. 12B shows the results of real time polymerase chain reaction (PCR) showing TGM-2 transcript levels in murine sclera of myopic mice compared to that of the control mice. The TGM-2 transcript level in myopic mice is increased by approximately 2.5 folds compared to that of the control mice. Error bars=SD. Student T test for independent samples p<0.05.

FIG. 12C shows a Western Blot image detecting the relative protein levels of TG-2 (top) and tubulin loading control (bottom) in primary fibroblasts cultured from murine sclera treated with lens (“myopia”) and control. The primary scleral fibroblasts treated with lens (“myopia”) show increase in TG-2 protein level compared to that of the control.

FIG. 12D shows a Western Blot image detecting the relative protein levels of TG-2 (top) and tubulin loading control (bottom) in M₂^{-/-} and M₅^{-/-} knock-out and M₂^{+/+} and M₅^{+/+} wild type mice sclera. It is observed that the TG-2 protein level in the scleral tissue of M₂^{-/-} murine eyes was reduced whereas the TG-2 protein level in the scleral tissue of M₅^{-/-} murine eyes was increased, as compared to the respective wild type.

FIG. 12E shows the real time PCR result illustrating relative TGM-2 transcript levels in M₁^{-/-}, M₂^{-/-}, M₃^{-/-} and M₅^{-/-} knock-out and M₁^{+/+}, M₂^{+/+}, M₃^{+/+} and M₅^{+/+} wild type mice sclera. GAPDH was used as an internal control and the message level was normalized with GAPDH house keeping gene. Error bars=SD. ANOVA T test p<0.05. It is observed that the TGM-2 transcript level in the scleral tissue of M₃^{-/-} murine eyes was most significantly reduced compared to its wild type.

FIG. 13A shows the refractive errors of homozygous TGM-2-deleted mice and wild-type mice measured at 2, 4 and 6 weeks. Myopia induction was performed using uniocular −10 diopter negative lenses (indicated as “minus lens treated eye”) in wild-type and homozygous TGM2-deleted mice. Refractive errors were determined by infrared photorefraction. Positive and negative spherical equivalents represent hyperopic and myopic refractive errors respectively. Upon myopia induction with negative lens, the homozygous TGM2-deleted mice remained hyperopic at week 8 (indicated as 6 weeks after induction) compared to wild type mice. Height of bars or symbols represents mean and error bars SD. *: p<0.05 and **: p<0.001.

FIG. 13B shows the axial length measurement of homozygous TGM-2-deleted mice and wild-type mice at 2, 4 and 6 weeks. Axial length was determined by optical coherence interferometry. Axial length elongation was significantly higher in negative lens treated wild type mice at week 8 (indicated as 6 weeks after induction), but not in TGM2-deleted mice. Height of bars or symbols represents mean and error bars SD. *: p<0.05 and **: p<0.001.

FIG. 13C shows TGM-2 transcript level of murine scleral derived fibroblasts when treated to exogenous atropine or carbachol at different concentrations. Murine scleral derived fibroblasts were cultured to passage 2 and treated to exogenous atropine or carbachol at 0.01 μM, 0.1 μM, 1 μM, 5 μM and 10 μM for 5 days. Following 5 days of treatment, the total RNA was extracted from these cells and RT-qPCR performed to determine transcript levels of TGM-2. The levels were normalized to GAPDH internal control. In sclera-derived fibroblasts from wild-type mice, treatment with the pan-muscarinic antagonist atropine induced a reduction in the level of TGM-2 transcript, whereas the pan-muscarinic agonist carbachol had the opposite effect. Height of bars or symbols represents mean and error bars SD. *: p<0.05 and **: p<0.001.

FIG. 13D shows a Western Blot image detecting the TG-2 protein level extracted from murine scleral derived fibroblasts of FIG. 13C. Total protein was extracted from the cultured cells as described in FIG. 13C and TG-2 proteins detected (top and middle) using primary antibody (Ab421) specific for TG2. β-tubulin was used as a loading control for protein. It can be observed that treatment with atropine induced a reduction in the TG-2 protein level in sclera-derived fibroblasts of wild-type mice, whereas the pan-muscarinic agonist carbachol had the opposite effect.

FIG. 13E shows a plot illustrating the results of the transamidase activity of TG-2 extracted from the murine scleral derived fibroblasts of FIG. 13C. Treatment with atropine induced a reduction in transamidase activity of TG-2 whereas the pan-muscarinic agonist had opposite effects. Height of bars or symbols represents mean and error bars SD. *: p<0.05 and **: p<0.001.

FIG. 14A shows the axial length measured in mice eyes that were treated with different muscarinic antagonists. Mouse eyes were treated with −15D spectacle lens, lens with 0.1% himbacine, lens with 0.1% darifenacin, lens with 0.1% 4-DAMP and lens with 0.1% atropine for 2 weeks and 4 weeks (n=6 mice in each group). Right eyes were experimental and left eyes served as contra-lateral control. Axial length was measured at 2 weeks and 4 weeks after treatment. The axial length was significantly reduced in the drug treated eyes as compared to control and lens treated eyes. Among all the drugs, himbacine showed the most significant amount of reduction in the mouse myopia progression.

FIG. 14B shows the refractive error measurements of mice eyes that were treated with different muscarinic antagonists as described in FIG. 16A. Automated infra-red photorefractor was used to measure the refractive error measurements. The refractive error was shifted from myopic to hyperoic after receiving the drugs.

FIG. 15 shows a schematic diagram illustrating biological processes regulated by TGM-2 and TGM-3 pathway, analyzed using Pathway Studio 5.0. TGM-2 plays a central role in wound healing, matrix remodeling and apoptosis.

FIG. 16 shows the knockdown efficiency of siRNA against TG-2 in human scleral fibroblasts (HSF) compared against untreated HSFs and a negative control.

FIG. 17A shows cell growth and proliferation assessed in real time using impedance technology via XCELLigence system RTCA SP (Roche Applied Science, IN) for HSF treated with TG-2-siRNA, untreated cells and a negative control.

FIG. 17B shows the doubling time of TG-2-siRNA treated HSF compared to untreated HSFs and a negative control.

FIG. 18 shows a schematic diagram illustrating biological processes regulated by TG-2 and TG-3 as well as interactions between TG-2 and muscarinic receptor pathway, analyzed using Pathway Studio 6.0. Interaction between TG-2 and muscarinic receptors are involved through Mitogen-Activated Protein Kinase (MAPK) pathway.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

The present invention is based on the surprising finding that transglutaminases, in particular tissue transglutaminase (TG-2), a molecule previously implicated in wound healing and migration of cells, are able to mediate formation of diseases or disorder associated with the expression of the TG-2 gene, including myopia. For example, TG-2 gene or protein or both was up-regulated in myopic murine sclera compared to control and that atropine, an anti-muscarinic drug down-regulated the gene. Thus, the present invention relates to a method of treating a disease or disorder associated with the expression of transglutaminase-2 (TGM-2). The method further includes administering to a subject a TG-2 expression inhibitor. The TG-2 inhibitor can be one of darifenacin or an analogue thereof; 1,1-Dimethyl-4-diphenylacetoxypiperidinium iodide (4-DAMP) or an analogue thereof; a nucleic acid molecule that inhibits expression of TGM-2; and himbacine or an analogue thereof. The analogue of himbacine can comprise a compound of Formula I:

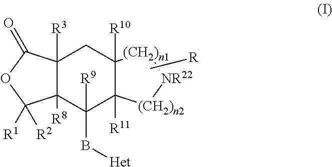

or a pharmaceutically acceptable salt thereof
wherein:

R is 1 to 3 substituents independently selected from the group consisting of H, C₁-C₆ alkyl, halogen, hydroxy, amino, (C₁-C₆)alkyl-amino, (C₁-C₆)-dialkylamino, (C₁-C₆)alkoxy, —COR¹⁶, —COOR¹⁷, —SOR¹⁶, —SO₂R¹⁶, —SO₂NR NR¹⁷R¹⁸, —NR¹⁷SO₂R₁₈, NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, C₃-C₆cycloalkyl, aryl(C₁-C₆)alkyl, hydroxy(C₁-C₆)alkyl, amino-(C₁-C₆)-alkyl, aryl and thio(C₁-C₆)alkyl;

R¹ and R² are independently selected from the group consisting of H, C₁-C₆ alkyl, fluoro(C₁-C₆)alkyl, difluoro(C₁-C₆)alkyl, trifluoro-(C₁-C₆)alkyl, C₃-C₆cycloalkyl, C₂-C₆ alkenyl, aryl(C₁-C₆)alkyl, hydroxy-(C₁-C₆)alkyl, amino(C₁-C₆)alkyl, aryl and thio(C₁-C₆)alkyl; or and R¹ and R² together form an ═O group;

R³ is H, hydroxy, C₁-C₆alkoxy, aryloxy, aryl(C₁-C₆)alkyloxy, heteroaryloxy, heteroaryl(C₁-C₆)alkyloxy, (C₃-C₆)cycloalkyloxy, —SOR¹⁶, —SO₂R¹⁷, —SO₂NR¹⁸R¹⁹, —SR¹⁸, —SO₃H, —C(O)OR¹⁷, —C(O)NR¹⁸R¹⁹, —OC(O)R³², —OC(O)NR³³R³⁴, —(CR³³R³⁴)ₙOR³², —NR⁴R⁵, —NR³³COOR³², —NR³³COR³², —NR³³S(O)₂R³², —NR³³CONR³³R³⁴, —NR³³S(O)₂NR³³R³⁴, —(CR³³R³⁴)ₙNR⁴R⁵, —(CR³³R³⁴)ₙNR³³COR³², —(CR³³R³⁴)ₙNR³³COR³², —(CR³³R³⁴)ₙNR³³S(O)₂R³², —(CR³³R³⁴)ₙNR³³CONR³³R³⁴, —(CR³³R³⁴)ₙNR^{33s}(O)₂NR³³R³⁴, (C₁-C₆)alkyl, halogen, C₃-C₆cycloalkyl, C₂-C₆alkenyl, —CN, aryl, heteroaryl, heterocycloalkyl, —P(O)(OR⁷)₂ or (C₁-C₆)alkyl substituted by 1 to 3 substituents independently selected from the group consisting of halogen, —OH, —NH₂, aryl, —COOH, —SO₃H, thio and (C₁-C₆)alkylthio;

n is 1, 2, 3 or 4;

n1 and n2 are independently 0-3, provided both are not 0;

Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of C₁-C₆alkyl; —NR⁴R⁵; —NHCOR²⁶; —NHSO₂R¹⁶; R²¹-aryl; aryl wherein adjacent carbons form a ring with a methylenedioxy group; and R²¹-heteroaryl;

R⁴ and R⁵ are independently selected from the group consisting of H, C₁-C₆ alkyl, phenyl, benzyl and C₃-C₆ cycloalkyl, or R⁴ and R⁵ together are —(CH₂)₃—, —(CH₂)₄—, —(CH₂)₅— or —(CH₂)₂NR⁷—(CH₂)₂— and form a ring with the nitrogen to which they are attached;

R⁷ is H or (C₁-C₆)alkyl;

R⁸, R¹⁰ and R¹¹ are independently selected from the group consisting of R¹ and —OR¹;

R⁹ is H, OH, —NR⁴R⁵, C₁-C₆alkoxy, halogen or halo(C₁-C₆)alkyl;

B is —(CH₂)n₃- or cis or trans —(CH₂)n₄CR¹²═CR^{12a}(CH₂)n₅, wherein n₃ is 0-5, n₄ and n₅ are independently 0-2, and R¹² and R^{12a} are independently selected from the group consisting of H, C₁-C₆ alkyl and halogen;

R¹⁶ and R^{16a} are independently selected from the group consisting of C₁-C₆ alkyl, phenyl and benzyl;

R¹⁷, R¹⁸ and R¹⁹ are independently selected from the group consisting of H, C₁-C₆alkyl, phenyl and benzyl;

R²¹ is 1 to 3 substituents independently selected from the group consisting of H, —CF₃, —OCF₃, halogen, —NO₂, —CN, C₁-C₆alkyl, C₁-C₆alkoxy, —NH₂, (C₁-C₆)-alkyl-amino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —CONR²⁴R²⁵, —NHCOR¹⁶, —NHSO₂R¹⁶, —NHSO₂CH₂CF₃, —SO₂NR²⁴R²⁵, —NR²⁹C(O)NR²⁴R²⁵, —SO₂R³⁰, —P(O)(OR²⁹)₂, aryl, aryl(C₁-C₆)alkyl, heteroaryl, heterocycloalkyl, and —CR²⁹(═NOR²⁸);

R²² is —COR²³, —S(O)R³¹, —S(O)₂R³¹, —SO₂NR²⁴R²⁵ or —COOR²⁷;

R²³ is halo (C₁-C₆)alkyl; C₂-C₆alkenyl; halo (C₂-C₆)alkenyl; C₂-C₆alkynyl; C₃-C₇-cycloalkyl; (C₃-C₇)cycloalkyl(C₁-C₆)alkyl; (C₃-C₇)cycloalkyl substituted by 1 to 3 substituents selected from the group consisting of halo, (C₁-C₃)alkoxy(C₁-C₃)alkyl, hydroxy and C₁-C₆alkoxy; aryl; aryl(C₂-C₆)alkyl; heteroaryl; heterocycloalkyl; (C₁-C₆)alkyl substituted by 1-3 substituents independently selected from —COOH and —SO₃H; or

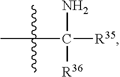

wherein R³⁵ and R³⁶ are independently selected from the group consisting of H, alkyl, or R³⁷-substituted C₁-C₆alkyl, wherein R³⁷ is selected from the group consisting of HO—, HS—, CH₂S—, —NH₂, phenyl, p-hydroxyphenyl and indolyl;

R²⁴ and R²⁵ are independently selected form the group consisting of H, C₁-C₆alkyl, halo(C₁-C₆)alkyl, C₂-C₆alkenyl, halo(C₂-C₆)alkyl, C₂-C₆alkynyl, aryl, aryl-(C₁-C₆)alkyl, C₃-C₇-cycloalkyl, halo(C₃-C₇)cycloalkyl, (C₁-C₃)alkoxy(C₁-C₃)-alkyl, hydroxy and C₁-C₆alkoxy;

R²⁶ is C₃-C₇-cycloalkyl, aryl, aryl-(C₁-C₆)alkyl, heteroaryl, heteroaryl-(C₁-C₆)alkyl or (C₁-C₆)alkylamino;

R²⁷ is C₁-C₆alkyl, phenyl, benzyl, (C₁-C₃)alkoxy(C₁-C₃)-alkyl, (C₃-C₇)-cycloalkyl, carboxy(C₁-C₆)alkyl, sulfo(C₁-C₆)alkyl, or (C₁-C₆)alkyl substituted by NR¹⁸R¹⁹ and carboxy;

R²⁸ is H, C₁-C₆alkyl, phenyl, benzyl or (C₁-C₆)alkoxy(C₁-C₃)alkyl;

R²⁹ and R³⁰ are independently selected from the group consisting of H and C₁-C₆alkyl;

R³¹ is (C₁-C₆)alkyl; halo(C₁-C₆)alkyl; C₂-C₆alkenyl; halo(C₂-C₆)alkyl; C₂-C₆alkynyl; C₃-C₇-cycloalkyl; (C₃-C₇)cycloalkyl substituted by 1 to 3 substituents selected from the group consisting of halo, (C₁-C₃)alkoxy(C₁-C₃)alkyl, hydroxy and C₁-C₆alkoxy; aryl; aryl (C₁-C₆)alkyl; heteroaryl; heterocycloalkyl; (C₁-C₆)alkyl substituted by 1-3 substituents independently selected from —COOH and —SO₃H; or (C₁-C₆)alkoxy;

R³² is R³⁵—(C₁-C₆)alkyl, R³⁵—(C₃-C₇)cycloalkyl, R³⁵—(C₂-C₆)alkenyl, R³⁵—(C₂-C₆)-alkynyl or R³⁵-aryl, wherein R³⁵ is 1 or 2 substituents independently selected from the group consisting of H, —COOH, —NH₂, —SO₃H, ═O and ═NOR²⁸; and

R³³ and R³⁴ are independently selected from the group consisting of H, (C₁-C₆)alkyl and C₃-C₇-cycloalkyl.

In some embodiments, the TG-2 inhibitor does not include atropine.

In the context of various embodiments, the term “TG-2 inhibitor”, as used herein, refers to an agent that decreases activity of TG-2. This may, for example, be achieved by interfering with the catalytic activity of TG-2, e.g., by a competitive or allosteric inhibitor or an antibody, antibody fragment or antibody-like molecule. Alternatively, TG-2 activity may be inhibited by decreasing the levels of said protein. This may for example be done by targeting the protein for degradation or by interfering with the expression of the gene encoding TG-2. This gene is referred herein as TGM-2. Inhibition of the expression of the TGM-2 gene may for example be achieved by RNAi, siRNA or similar techniques. An inhibitor that blocks TG-2 expression would therefore be a TGM-2 inhibitor as well.

“TG-2”, as used herein, refers to tissue transglutaminase 2, an enzyme (EC 2.3.2.13) that crosslinks proteins between an ε-amino group of a lysine residue and a γ-carboxamide group of glutamine residue, creating an inter- or intramolecular bond that is highly resistant to proteolysis (protein degradation). In various embodiments, the term relates to human TG-2 with the UniProt Accession No. P21980 (UniProtKB/Swiss-Prot Databank, Version 139, 25 Jan. 2012), including the three known splicing isoforms P21980-1, P21980-2 and P21980-3.

The term “TGM-2”, as used herein, refers to the gene encoding TG-2, as defined herein. In various embodiments, the gene is the human TGM-2 gene with the GenBank Accession No. NM_{—}004613 (Version NM_{—}004613.2) or NM 198951 (Version NM_{—}198951.1).

The term “analogue” used in relation to a TG-2 inhibitor described herein refers to a compound that is structurally similar to the TG-2 inhibitor and exhibits similar pharmaceutical activity.

In some embodiments, the method of treating a disease or disorder associated with the expression of TGM-2 comprises administering to a subject a TG-2 inhibitor having the following formula

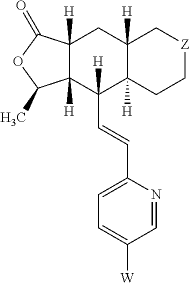

wherein W and Z are defined in the following table:

| W | Z |
|---|---|
| 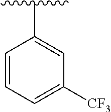 [SMILES: *c1cccc(C(F)(F)F)c1] | —S— |
| 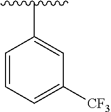 [SMILES: *c1cccc(C(F)(F)F)c1] | —S(O)— |
| 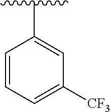 [SMILES: *c1cccc(C(F)(F)F)c1] | 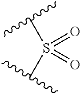 [SMILES: *S(*)(=O)=O] |
| 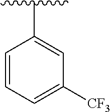 [SMILES: *c1cccc(C(F)(F)F)c1] | —O— |
| 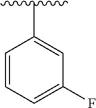 [SMILES: *c1cccc(F)c1] | —O— |
| 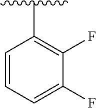 [SMILES: *c1cccc(F)c1F] | —O— |
| 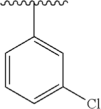 [SMILES: *c1cccc(Cl)c1] | —O— |
| 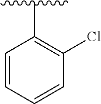 [SMILES: *c1ccccc1Cl] | —O— |
| 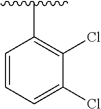 [SMILES: *c1cccc(Cl)c1Cl] | —O— |
| 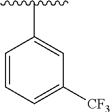 [SMILES: *c1cccc(C(F)(F)F)c1] | 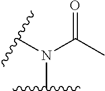 [SMILES: *N(*)C(C)=O] |
| 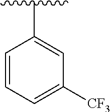 [SMILES: *c1cccc(C(F)(F)F)c1] | 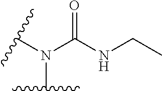 [SMILES: *N(*)C(=O)NCC] |
| 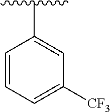 [SMILES: *c1cccc(C(F)(F)F)c1] | 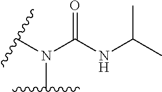 [SMILES: *N(*)C(=O)NC(C)C] |
| 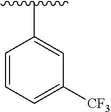 [SMILES: *c1cccc(C(F)(F)F)c1] | 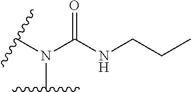 [SMILES: *N(*)C(=O)NCCC] |
| 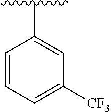 [SMILES: *c1cccc(C(F)(F)F)c1] | 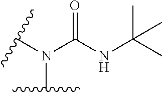 [SMILES: *N(*)C(=O)NC(C)(C)C] |
| 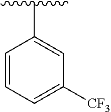 [SMILES: *c1cccc(C(F)(F)F)c1] | —NH— |
| 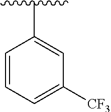 [SMILES: *c1cccc(C(F)(F)F)c1] | 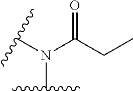 [SMILES: *N(*)C(=O)CC] |
| 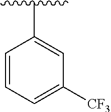 [SMILES: *c1cccc(C(F)(F)F)c1] | 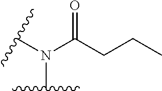 [SMILES: *N(*)C(=O)CCC] |
| 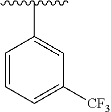 [SMILES: *c1cccc(C(F)(F)F)c1] | 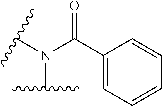 [SMILES: *N(*)C(=O)c1ccccc1] |
| 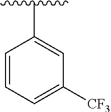 [SMILES: *c1cccc(C(F)(F)F)c1] | 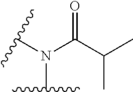 [SMILES: *N(*)C(=O)C(C)C] |
| 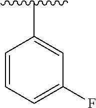 [SMILES: *c1cccc(F)c1] | 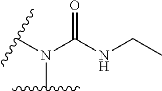 [SMILES: *N(*)C(=O)NCC] |
| 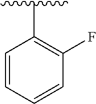 [SMILES: *c1ccccc1F] | 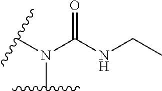 [SMILES: *N(*)C(=O)NCC] |
| 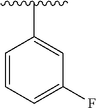 [SMILES: *c1cccc(F)c1] | 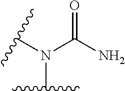 [SMILES: *N(*)C(N)=O] |
| 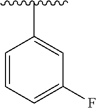 [SMILES: *c1cccc(F)c1] | 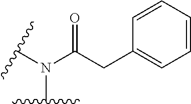 [SMILES: *N(*)C(=O)Cc1ccccc1] |
| 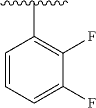 [SMILES: *c1cccc(F)c1F] | 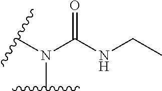 [SMILES: *N(*)C(=O)NCC] |
| 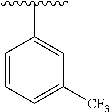 [SMILES: *c1cccc(C(F)(F)F)c1] | —N(CH₃)— |

In the compound of Formula I, the term “alkyl”, alone or in combination, refers to a fully saturated aliphatic hydrocarbon such as a straight or branched chain hydrocarbon group. The alkyl can for example be optionally substituted. In certain embodiments, an alkyl can comprise 1 to 6 carbon atoms, 1 to 5 carbon atoms, 1 to 4 carbon atoms, or 1 to 3 carbon atoms, wherein (whenever it appears herein in any of the definitions given below) a numerical range, such as “1 to 6” or “C₁-C₆”, refers to each integer in the given range, e.g. “C₁-C₆ alkyl” means that an alkyl group comprising only 1 carbon atom, 2 carbon atoms, 3 carbon atoms, etc., up to and including 6 carbon atoms. Examples of alkyl groups include, but are not limited to, methyl, ethyl, n-propyl, isopropyl, n-butyl, isobutyl, sec-butyl, tert-butyl, tert-amyl, pentyl, hexyl, and the like. Fluoroalkyl, difluoroalkyl and trifluoroalkyl mean alkyl chains wherein the terminal carbon is substituted by 1, 2 or 3 fluoroatoms, e.g., —CF₃, —CH₂CF₃, —CH₂CHF₂ or —CH₂CH₂F. Haloalkyl means an alkyl chain substituted by 1 to 3 halo atoms.

The term “alkenyl” as used herein refers to an alkyl group that contains in the straight or branched hydrocarbon chain one or more double bonds. In certain embodiments, an alkenyl comprises 2 to 6 carbon atoms, for example 2 to 5 carbon atoms or 2 to 4 carbon atoms, wherein a numerical range, such as “2 to 6” or “C₂-C₆”, refers to each integer in the given range, e.g. “C₂-C₆ alkenyl” means that an alkenyl group comprising 2 carbon atoms, 3 carbon atoms, etc., up to and including 6 carbon atoms. An alkenyl used in this invention can for example be optionally substituted. Examples of alkenyl groups include, but are not limited to, ethenyl, propenyl, butenyl, 1,4-butadienyl, pentenyl, 4-methylhex-1-enyl, 4-ethyl-2-methylhex-1-enyl and the like.

The term “alkynyl” as used herein refers to an alkyl group that contains in the straight or branched hydrocarbon chain one or more triple bonds. In certain embodiments, an alkynyl comprises 2 to 6 carbon atoms, for example 2 to 6 carbon atoms, 2 to 5 carbon atoms, or 2 to 4 carbon atoms, wherein a numerical range, such as “2 to 6” or “C₂-C₆”, refers to each integer in the given range, e.g. “C₂-C₆ alkynyl” means that an alkynyl group comprising 2 carbon atoms, 3 carbon atoms, etc., up to and including 6 carbon atoms. An alkynyl group of this invention may be optionally substituted. Examples of alkyne groups include, but are not limited to, ethynyl, propynyl, butynyl, and the like. Where an alkyl, alkenyl or alkynyl chain joins two other variables and is therefore bivalent, the terms alkylene, alkenylene and alkynylene are used. Haloalkenyl means an alkenyl chain substituted by 1 to 3 halo atoms.

The term “cycloalkyl” refers to a completely saturated hydrocarbon ring. The cycloalkyl group used in this invention may range from C₃ to C₈ or C₃ to C₆. A cycloalkyl group of this invention can for example be optionally substituted. Examples of cycloalkyl groups include, but are not limited to cyclopropyl, cyclobutyl, cyclopentyl, cyclohexyl and the like.

The term “heterocycloalkyl” as a substituent on Het means saturated rings of 4 to 7 atoms comprised of 3 to 4 carbon atoms and 1 to 3 heteroatoms selected from the group consisting of —O—, —S— and —NR⁷— joined to the rest of the molecule through a carbon atom. Examples of heterocyclo-alkyl groups are 2-azetidinyl, 2-pyrrolidinyl, tetrahydrothiophen-2-yl, tetrahydro-2-furanyl, 4-piperidinyl, 2-piperazinyl, tetrahydro-4-pyranyl, 2-morpholinyl and 2-thiomorpholinyl.

“Halogen” refers to fluorine, chlorine, bromine or iodine radicals.

When R⁴ and R⁵ join to form a ring with the nitrogen to which they are attached, the rings formed are 1-pyrrolidinyl, 1-piperidinyl and 1-piperazinyl, wherein the piperazinyl ring may also be substituted at the 4-position nitrogen by a group R⁷.

“Dihydroxy(C₁-C₆)alkyl” refers to an alkyl chain substituted by two hydroxyl groups on two different carbon atoms.

The term “aryl” refers to an aromatic ring wherein each of the atoms forming the ring is a carbon atom. Aryl rings may be formed by five, six, seven, eight, nine, or more than nine carbon atoms. Aryl groups may be optionally substituted and may mean phenyl, naphthyl, indenyl, tetrahydronaphthyl or indanyl.

The term “optionally substituted” refers to a group in which none, one, or more than one of the hydrogen atoms has been replaced with one or more group(s) are independently selected from: alkyl, heteroalkyl, haloalkyl, heterohaloalkyl, cycloalkyl, aryl, arylalkyl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups.

The term “heteroaryl” means a single ring, bicyclic or benzofused heteroaromatic group of 5 to 10 atoms comprised of 2 to 9 carbon atoms and 1 to 4 heteroatoms independently selected from the group consisting of N, O and S, provided that the rings do not include adjacent oxygen and/or sulphur atoms. N-oxides of the ring nitrogens are also included, as well as compounds wherein a ring nitrogen is substituted by a C₁-C₄ alkyl group to form a quaternary amine. Examples of single-ring heteroaryl groups are pyridyl, oxazolyl, isoxazoyl, oxadiazolyl, furanyl, pyrrolyl, thienyl, imidazolyl, pyrazolyl, tetrazolyl, thiazolyl, isothiazolyl, thiadiazolyl, pyrazinyl, pyrimidyl, pyridazinyl and triazolyl. Examples of bicyclic heteroaryl groups are naphthyridyl (e.g., 1, 5 or 1, 6), imidazopyridyl, pyrido[2,3]imidazolyl, pyridopyrimidinyl and 7-azaindolyl. Examples of benzofused heteroaryl groups are indolyl, quinolyl, isoquinolyl, phthalazinyl, benzothienyl (i.e., thionaphthenyl), benzimidazolyl, benzofunanyl, benzoxazolyl and benzofurazanyl. All positional isomers are contemplated, e.g. 1-pyridyl, 2-pyridyl, 3-pyridyl and 4-pyridyl. W-substituted heteroaryl refers to such groups wherein substitutable ring carbon atoms have a substituent as defined above, or where adjacent carbon atoms form a ring with an alkylene group or a methylenedioxy group.

The term “Het” is exemplified by the single ring, bicyclic and benzofused heteroaryl groups as defined immediately above, as well as tricyclic groups such as benzoquinolinyl (e.g., 1, 4 or 7,8) or phenanthrolinyl (e.g., 1,7; 1,10; or 4,7). Het groups are joined to group B by a carbon ring member, e.g. Het is 2-pyridyl, 3-pyridyl or 2-quinolyl.

Examples of heteroaryl groups wherein adjacent carbon atoms form a ring with an alkylene group are 2,3-cyclopentenopyridine, 2,3-cyclohexenopyridine and 2,3-cyclopentenopyrine. When R²² is —COR²³ and R²³ is

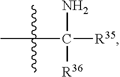

this group is an acyl radical of an amino acid.

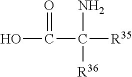

is a naturally occurring amino acid selected from alanine, glycine, valine, leucine, isoleucine, phenylalanine, tryptophan, methionine, serine, theronin, cysteine, cystine or tyrosine.

The above statements, wherein, for example, R⁴ and R⁵ are said to be independently selected from a group of substituents, means that R⁴ and R⁵ are independently selected, but also that where an R⁴ and R⁵ variable occurs more than once in a molecule, those occurrences are independently selected. Those skilled in the art will recognize that the size and nature of the substituent(s) will affect the number of substituents which can be present.

The compounds of Formula I as described herein have at least one asymmetrical carbon atom and therefore all isomers, including diastereomers and rotational isomers are contemplated. The compounds include (+)- and (−)-isomers in both pure form and in admixture, including racemic mixtures. Isomers can be prepared using conventional techniques, either by reacting optically pure or optically enriched starting materials or by separating isomers of a compound of formula I.

In some embodiments, a TG-2 inhibitor that can be used in the methods of the present invention can have the following stereochemistry:

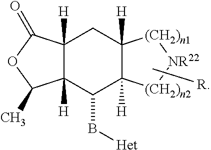

The TG-2 inhibitor used in the present invention with a basic group can encompass any pharmaceutically acceptable salts, esters, salts of such esters or any other compound, which upon administration into a subject, including a human, is capable of providing the biologically active metabolite or residue thereof. Accordingly, also described herein is drawn to prodrugs and pharmaceutically acceptable salts of such pro-drugs, and other bioequivalents. The term “pharmaceutically acceptable salt” refers to physiologically and pharmaceutically acceptable salt(s) of the TG-2 inhibitors described herein; i.e. salts that retain the desired biological activity of the parent compound and do not impart undesired toxicological effects thereto. Examples of suitable pharmaceutically acceptable salts include but are not limited to (a) salts formed with cations such as sodium, potassium, ammonium, magnesium, calcium, polyamines such as spermine and spermidine, etc; (b) acid addition salts formed with inorganic acids for example hydrochloric acid, sulfuric acid, phosphoric acid, nitric acid and the like; (c) salts formed with organic acids such as, for example, acetic acid, citric acid, oxalic acid, malonic, salicylic acid, malic acid, fumaric acid, succinic acid, ascorbic acid, maleic acid, methanesulfonic acid and other mineral and carboxylic acids well known to those in the art. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for the purposes of the invention.

Certain TG-2 inhibitors used in the present invention can be acidic (e.g., those compounds which possess a carboxyl group). These TG-2 inhibitors form pharmaceutical acceptable salts with inorganic and organic bases. Examples of such salts are the sodium, potassium, calcium, aluminum, lithium, gold and silver salts. Also included are salts formed with pharmaceutically acceptable amines such as ammonia, alkyl amines, hydroxyalkylamines, N-methylglucamine and the like.

The TG-2 inhibitor used in the present invention can be a nucleic acid molecule that inhibits expression of TGM-2. The term “nucleic acid molecule” used in the invention can refer to any nucleic acid molecule in any possible configuration, such as a linearized single stranded, a double stranded or a combination thereof, as long as it is capable of inhibiting the expression of TGM-2. Examples of such nucleic acid molecules may include, but are not limited to DNA molecules (e.g., cDNA or genomic DNA) or RNA molecules (e.g., mRNA). DNA or RNA may be of genomic or synthetic origin and may be single or double stranded. The nucleic acid molecule can also be an aptamer, an antisense RNA, small interfering (siRNA), micro RNA (miRNA), analogues of the DNA or RNA generated using nucleotide analogues or using nucleic acid chemistry, cDNA synthetic DNA, a copolymer of DNA and RNA, oligonucleotides, PNA (protein nucleic acids) or combinations thereof. A respective nucleic acid may furthermore contain non-natural nucleotide analogues and/or be linked to an affinity tag or a label.

The TG-2 inhibitors used in the present invention can generally be prepared by processes known in the art, as described in for example PCT Application No. PCT/US03/32936.

The term “treat” or “treating” as used herein is intended to refer to providing an pharmaceutically effective amount of a TG-2 inhibitor as described herein or a respective pharmaceutical composition or medicament thereof sufficient to act prophylactically to prevent the development of a weakened and/or unhealthy state; and/or providing a subject with a sufficient amount of the inhibitor or pharmaceutical composition or medicament thereof so as to alleviate or eliminate a disease state and/or the symptoms of a disease state, and a weakened and/or unhealthy state.

In some embodiments, the TG-2 inhibitors described herein can target the respective TG-2 protein and/or the expression of TGM-2. In other embodiments, the TG-2 inhibitors can further target at least one muscarinic receptor, for example M₂ or M₃ or inhibit the expression of at least one muscarinic receptor for example, M₂ or M₃. As a non-limiting illustrative example, the binding selectivity of himbacine to muscarinic receptor M₂ was about 80%. Furthermore, himbacine bound to all five cloned receptor subtypes in the order to potencies: hM₂=hM₄>hM₃>hM₁>hM₅ (Kd values were 4, 7, 59, 83 and 296 nM, respectively) (data not shown). Therefore, without wishing to be bound by theory, himbacine appears to be a potent muscarinic antagonist that displays high selectivity for M₂ or M₄ receptors, as compared to M₁ or M₃ receptors.

In some embodiments, the TG-2 inhibitor can be administered into the subject in the range of about 0.01 μM to about 100 μM; about 0.01 μM to about 80 μM; about 0.01 μM to about 50 μM; about 0.01 μM to about 30 μM; about 0.01 μM to about 20 μM; about 0.01 μM to about 10 μM; about 0.01 μM to about 5 μM; about 0.01 μM to about 4 μM; about 0.01 μM to about 3 μM; about 0.01 μM to about 2 μM; about 0.01 μM to about 1 μM; about 0.01 μM to about 0.5 μM; about 0.01 μM to about 0.1 μM; about 0.1 μM to about 100 μM; about 1 μM to about 100 μM; about 1 μM to about 50 μM; about 1 μM to about 10 μM; about 1.5 μM to about 100 μM; or about 2 μM to about 100 μM. In particular embodiments, about 0.01 μM to about 1 μM of the TG-2 inhibitor is administered into the subject. In this context, without wishing to be bound by theory, it is found that a lower concentration of about 0.01 μM to about 10 μM; or about 0.01 μM to about 5 μM; or about 0.01 μM to about 1 μM of himbacine can sufficiently reduce TG2 activity for example, as compared to that of atropine.

In this context, the subject can be in the age of between 3 years old to 17 years old; between 3 years old to 14 years old; between 3 years old to 10 years old; between 3 years old to 7 years old; between 4 years old to 17 years old; between 4 years old to 14 years old; between 6 years old to 10 years old; or between 7 years old to 17 years old.

In some embodiments, the subject can be a mammal. In other embodiments, the subject can be a human. The mammal may include a Rodentia, Canis, Ungulate, Felidae, Leporidae, and Macaque. Examples of a rodent include, but are not limited to, a mouse, rat, squirrel, chipmunk, gopher, porcupine, beaver, hamster, gerbil, guinea pig, chinchilla, prairie dog, and groundhog. Examples of Canis include, but are not limited to a dog, wolf, coyote and jackal. Examples of Ungulate include, but are not limited to a horse, donkey, zebra, sheep, pig, goat, camel, giraffe and moose. Examples of Felidae include, but are not limited to a cat, caracal, cougar, cheetah and leopard. Examples of Leporidae include, but are not limited to a rabbit, hare and jackrabbit. An example of a Macaque includes a rhesus monkey.

The TG-2 inhibitor as described herein can be formulated into a pharmaceutical composition. The pharmaceutical composition further includes a pharmaceutically acceptable carrier or excipient. The “carrier” or “excipient” can include any pharmaceutically acceptable carrier as long as the carrier is compatible with other ingredients of the formulation and not injurious to the patient. Accordingly, pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. The pharmaceutically acceptable carrier or excipient can be any of cellulose, hydroxymethylcellulose, cellulose acetate phthalate (CAP) gellan gum, polyalcohol, polyvinyl alcohol, hyaluronic acid, polyacrylic acid, carbopol polymer, poloxamer, poly(oxyethylene) and poly(oxypropylene) and block copolymers thereof, polyethylene oxide, polycarbophil, chitosan, cyclodextrin, liposome, nanoparticle, microparticle including microsphere and nanosphere, an ocular insert, ocular disc, soft contact lens, niosome, pharmacosome, collagen shield, ocular film or combinations thereof.

The TG-2 inhibitor or its pharmaceutical composition thereof can be administered into the subject via any suitable means as long as the intended therapeutic effect is achieved. In some embodiments, the pharmaceutical composition can for example be administered into the subject via topical or intra-ocular or systemic or oral, or rectal or transmucosal, or intestinal or intramuscular, or subcutaneous, or intramedullar, or intrathecal, or direct intraventricular, or intravenous, or intravitreal, or intraperitoneal, or intranasally administration.

In some embodiments, the disease or disorder associated with the expression of TGM-2 is myopia. Non-limiting examples of myopia can include axial myopia, refractive myopia, curvature myopia, index myopia, degenerative myopia, nocturnal myopia, pseudomyopia, induced myopia, form deprivation myopia, congenital myopia, youth onset myopia, school myopia, early adult onset myopia.

The present invention also relates to a method of identifying a candidate TG-2 inhibitor. The method includes a) contacting a TGM-2 expressing cell with a solution containing a putative TG-2 inhibitor to be identified and b) measuring whether the expression of TGM-2 is inhibited in the cell. Methods of determining the amount of a selected protein, for example TG-2, are well known in the art. A respective method may for instance rely on proteomics-based techniques. It may involve the use of an antibody, a fragment thereof or a proteinaceous binding molecule with antibody-like functions, which binds to the respective TG-2 protein. Methods of analyzing the expression of a protein are well established in the art. mRNA expression may for instance be quantified using northern blotting and in situ hybridization, RNAse protection assays and PCR-based methods. As a further example, an antibody, a fragment thereof or a proteinaceous binding molecule may be employed that can recognise specific duplexes, including DNA duplexes, RNA duplexes, and DNA-RNA hybrid duplexes or DNA-protein duplexes. Illustrative examples of methods of sequencing-based gene expression analysis include Serial Analysis of Gene Expression (SAGE), and gene expression analysis by massively parallel signature sequencing (MPSS).

PCR (polymerase chain reaction) is a standard technique useful to amplify and detect transcripts from a cell or from a tissue sample such as an eye tissue. Reverse Transcriptase PCR (RT-PCR), is for instance a sensitive quantitative method that can be used to compare mRNA levels in different samples (e.g., a TGM-2 expressing cell or respective tissue sample) to examine gene expression signatures. To perform RT-PCR, mRNA is isolated from a sample (e.g., total RNA isolated from an eye tissue sample for example). mRNA can be extracted, for example, from frozen or archived paraffin-embedded and fixed (e.g. formalin-fixed) tissue samples. Purification kits for RNA isolation are commercially available. The RT-PCR technique may also be used in the embodiment of real time quantitative PCR, which measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR. As a further example, the competitive PCR design may be used for gene expression analysis, possibly including automated, high-throughput matrix-assisted laser desorption ionization time-of-flight (MALDI-TOF) mass spectrometry detection and quantification of nucleic acid molecules. Further examples of techniques that may be included in PCR-based methods suitable for gene expression analysis may include, but are not limited to, differential display, amplified fragment length polymorphism (iAFLP), the BeadAmiy™ technology, BeadsArvay for Detection of Gene Expression (BADGE) and high coverage expression profiling (HiCEP) analysis. As a further example, determining gene expression of a tissue sample can also be performed with microarrays.

In some embodiments, the method of identifying a candidate TG-2 inhibitor can further include comparing the expression of TGM-2 obtained in step b) with that of a control measurement which was not exposed to the putative TG-2 inhibitor; and identifying the respective TG-2 inhibitor in the cell. A control measurement may include a TGM-2 expressing cell which was not exposed to the putative TG-2 inhibitor. In this context, detected levels may for example be compared to a control measurement or control level. The term “control level” as used herein refers to the number of molecules of the respective protein, e.g., in a cell, a mRNA or protein expression level of TG-2 protein, as well as to an activity level of a TG-2 protein in a control sample. The term can refer to a single reference measurement or to a plurality of reference measurements. In some embodiments, the control level may be a database of expression or activity values from previously conducted measurements.

In certain embodiments, determining an inhibited expression of TGM-2 in the TGM-2 expressing cell as compared to the control measurement may be an indication that a candidate TG-2 inhibitor is identified. In other embodiments, a candidate TG-2 inhibitor can also be identified by further determining the inhibited expression/activity of at least one muscarinic receptor M₂ or muscarinic receptor M₃ in the cell, relative to the control measurement.

According to the present invention, a gene expression level or an activity level or an amount of a protein, e.g. in a cell, is deemed to be “inhibited” when gene expression/activity/amount is decreased by about 10%, about 25%, about 50%, about 75%, about 100%, or higher, as compared to the control level. Alternatively, an expression level or an activity level is deemed “inhibited” or “decreased” when gene expression/or an activity is inhibited or decreased by at least about 0.1, at least about 0.2, at least about 1, at least about 2, at least about 5, or at least about 10 or more fold as compared to a control level.

In some embodiments, the cell is derived from a mammal as described herein. In other embodiments, the cell is derived from a human. In this context, the cell can be an epithelial cell, a subepithelial cell, a stromal cell, a cell membrane, an endothelial cell, a fibroblast cell or combinations thereof. The cell can also be derived from a tissue, for example an eye tissue or a part thereof. The eye tissue or part thereof can be sclera, cornea, retina pigment epithelium (RPE), choroid, conjunctiva or combinations thereof.

In order that the invention may be readily understood and put into practical effect, particular embodiments will now be described by way of examples and not limitations, and with reference to the figures.

### EXAMPLES

### Human and Animal Tissues

The present examples illustrate Donor human cadaver eyes (n=10) harvested within 24 hours from normal cadaver eyes (age range, 45-80 years) obtained at autopsy were provided by the Singapore Eye Bank and the protocol was approved by the Institutional Review Board of the Singapore Eye Research Institute which complies with the tenets of the Declaration of Helsinki. Mice (C57BL/6, n=122) were obtained from the animal holding unit of the National University of Singapore for culture work. Muscarinic receptor (M₁-M₅) mutant mice were obtained from Cell Signaling Laboratory at the National Cancer Institute at the National Institutes of Health in the USA. Heterozygous M₁-M₅-silenced mice were backcrossed ten generations to C57BL/6NTac to achieve genomic homogeneity of 99.95%, then crossbred in the animal holding unit of Singhealth Experimental Medical Center and genotyped for further studies. Heterozygous TGM2-silenced mice were backcrossed twelve generations to C57BL/6 to achieve genomic homogeneity of 99.95%, then crossbred in the animal holding unit of Singhealth Experimental Medical Center and genotyped. Naive control animals were housed in groups of 6 while experimental animals were housed individually after the age of 28 days at 25° C. on 12:12 hours of light: darkness, with mouse pellets and water available ad libitum. The approval was obtained from the Singhealth Institutional Animal Care and Use of Committee (IACUC; AALAC accredited) and all aspects of the study were in accordance with the Association for Research in Vision and Ophthalmology (ARVO) recommendations for animal experimentation.

### Data Analysis

Statistical comparisons between experimental groups were conducted using Student's t test or one-way ANOVA (Statistica 6.0, SPSS, Chicago, Ill., USA), followed by Tukey post hoc test. A significance level of p<0.05 was used. Data are presented as means±standard deviation.

### Example 1

### Animal Drug Treatment

The effects of muscarinic antagonists' treatment were examined using spectacle lens-induced myopia and were allocated to five groups: First group (n=12) received a daily 1 drop of 1% atropine sulfate (ATG), second group received a daily 1 drop of 1% himbacine (HBG), third group received a daily 1 drop of 1% 4-DAMP (DAG), fourth group received a daily 1 drop of 1% darifenacin (DFG) and the fifth group (n=12) received daily 1 drop of 0.9% of sterile normal saline (NSG) as a vehicle. Topical applications were administered to both eyes at the same time each day (approximately 9:00 AM) commencing on the 12^{th} day (initiation of spectacle lens treatment). A compatible drug level was determined prior to the in vivo use in a tissue culture study with mouse scleral fibroblasts. These concentrations [0.01%, 0.1%, 0.5% and 1%] were then tested in vivo in a small pilot study (data not shown). Results from the 0.1% drug treatment are being studied. The eyes were examined daily and no infections were found. This treatment schedule continued for four weeks starting on post-natal day 12 continued until post-natal day 42. All measurements were taken at post-natal day 42, the equivalent of 4 weeks of spectacle lens wear. Table 1 shows the subtypes and distribution of mAChRs in different tissues.

TABLE 1
Subtypes and distribution of mAChRs in different eye tissues
Muscarinic Acetylcholine Receptors in ocular tissues

| | Subtypes | | | | | | |
|---|---|---|---|---|---|---|---|
| Species | M₁ | M₂ | M₃ | M₄ | M₅ | Localization(s) | Remarks |
| Human | ▪ | ▪ | ▪ | ▪ | ▪ | Sclera, retinal | M₃ |
| | | | | | | pigment | predominates |
| | | | | | | epithelium, | in retina, |
| | | | | | | choroid | sclera, cornea, iris, |
| | | | | | | | ciliary |
| | | | | | | | body and |
| | | | | | | | lens |
| Mouse | ▪ | ▪ | ▪ | ▪ | ▪ | Retina, retinal | M₁ & M₄: |
| | | | | | | pigment | retina, |
| | | | | | | epithelium, | choroid, |
| | | | | | | choroids and | ciliary body |
| | | | | | | ciliary bodies, | and sclera |
| | | | | | | sclera | |
| Monkey | | ▪ | ▪ | ▪ | | Conjunctiva, | M₂-M₄: |
| | | | | | | meibomian | conjunctiva |
| | | | | | | glands, | M₃-M₄: |
| | | | | | | suprabasal | suprabasal |
| | | | | | | layers of the | layer of skin |
| | | | | | | skin epithelium | |
| Chick | | ▪ | ▪ | ▪ | | Retina, retinal | |
| | | | | | | pigment | |
| | | | | | | epithelium, | |
| | | | | | | choroid, and | |
| | | | | | | ciliary body | |
| Tree- | ▪ | ▪ | ▪ | ▪ | ▪ | Retina, | M₁ & M₄: |
| shrew | | | | | | choroid, sclera | retina, |
| | | | | | | and iris/ciliary | choroid, |
| | | | | | | body | ciliary body |
| | | | | | | | and sclera, |
| | | | | | | | M₂: ciliary |
| | | | | | | | body |
| | | | | | | | M₃ and M₅: |
| | | | | | | | all |
| Guinea | ▪ | ▪ | ▪ | ▪ | ▪ | Retina, | |
| pig | | | | | | choroid, sclera, | |
| | | | | | | and iris-ciliary | |
| | | | | | | body | |
| Bluegill | ▪ | ▪ | | | ▪ | Retinal | |
| Fish | | | | | | pigment | |
| | | | | | | epithelium | |

### Example 2

### Primary Cell Culture and Drug Treatment

Human scleral tissues (n=10) and eight-week old mouse sclera (n=100 eyes, 20 sclera/batch) from post-mortem eyes were obtained. The whole sclera was dissected very carefully and washed with cold phosphate buffered saline (PBS) three times. Fibrous sclera was placed in 60 mm culture dish with Dubelcco's Modified Eagle's Medium (DMEM, Gifco) supplemented with penicillin, streptomycin and amphotericin B and 10% Fetal Bovine Serum (FBS, Gifco). Tissue culture were incubated at 37° C., 5% CO₂ and allowed to reach 80% confluence. Cells were passaged sequentially by exposing cells to 0.25% Trypsin/0.5 mM EDTA at 37° C. for 5 minutes. The culture conditions were followed as described in Barathi, V. A., Weon, S. R., Beuerman, R. W., 2009b, “Expression of muscarinic receptors in human and mouse sclera and their role in the regulation of scleral fibroblasts proliferation”, Mol V is 15, 1277-1293.

All cells used in examples described herein were between passages 2-4. Passaged cells were plated at a concentration of 2×10⁵/mL into 6 well plates containing DMEM with 10% FBS. The cells were seen to attach to the bottom of the culture wells after 4 hours. Freshly prepared different muscarinic agents at different concentrations in DMEM with 10% FBS is shown in Table 2. Old media was removed from wells and 1 mL of drug was added with fresh full medium and incubated at 37° C. Fresh drugs were replaced daily consecutively for the next 5 days to avoid oxidation. The drugs/media was removed and wells were washed twice with 1×PBS. 200 μl of RIPA lysis buffer was added per well and incubated for 5 minutes and cells were scrapped into an eppendorf tube. The cells were stored at −20° C. until sonication. The cells were sonicated on ice thrice at 20% output power for 5 seconds each time and rested 1 minute in between each turn. Cell debris was spun down at 12000 rpm for 20 minutes. The supernatant was poured into a new eppendorf tube and cell concentrations were determined using the Dc Protein assay Kit (Bio-Rad, MA). Passaged cells were plated at a concentration of 1×10⁵ into 6 well plates containing DMEM with 10% FBS. The cells were seen to attach to the bottom of the culture wells after 4 hours. Freshly prepared muscarinic agents' at base line, 0.1, 1, 10 and 100 μM concentrations were added for 5 days (Table 2). The culture medium was removed and fresh medium containing the same muscarinic drug added every day to avoid drug changes such as atropine oxidation. Cell lysate was collected for relative quantitative (q) PCR and protein analysis at day 5.

TABLE 2
Concentrations of different muscarinic agents in DMEM with 10%
FBS for scleral fibroblast cells treatment.

| | Specificity | | | | | | | |
|---|---|---|---|---|---|---|---|---|
| Drugs/ | to | | | | | | | |
| Concentrations (μM) | mAChRs | 0 | 0.001 | 0.01 | 0.1 | 1 | 10 | 100 |
| Atropine | M1-M5 | X | | | X | X | X | X |
| Pirenzepine | M1 | X | | | X | X | X | X |
| Himbacine | M2/M4 | X | | | X | X | X | X |
| DAMP-4 | M3 | X | | | X | X | X | X |
| AFDX-116 | M2 | X | | | X | X | X | X |
| Tropicamide | M4 | X | X | X | X | X | | |
| Darifenacin | M3 | X | | X | X | X | X | |
| Hydrobromide | | | | | | | | |

### Example 3

### Sample Preparation

The cells were lysed by sonification in 1×RIPA lysis buffer (santa cruz biotechnology, Sc-24948) with 10 μl PMSF solution, 10 μl sodium orthovanadate solution and 20 μl protease inhibitor cocktail solution. After centrifugation at 20,000×g at 4° C. for 20 minutes, and the supernatant collected. The protein content in the supernatants was measured using the DC Protein Assay kit (Bio-Rad, MA) following the manufacturer's instructions. Samples were stored at −80° C. until assayed.

### Example 4

### Immunohistochemistry and Immunocytochemistry

The whole mouse eye (2 months old, n=6) and human sclera (n=3) were embedded in OCT (frozen tissue matrix) compound at −20° C. for 1 hour. Prepared tissue blocks were sectioned with cryostat at 5 microns thicknesses and collected on clean Polysine™ glass slides. Slides with the sections were air dried at room temperature for 1 hour and fixed with 4% para-formaldehyde for 10 minutes. After washing 3× with 1×PBS for 5 minutes, 4% goat serum diluted with 1×PBS was added as a blocking buffer. The slides were then covered and incubated for 1 hour at room temperature in a humid chamber. After rinsing with 1×PBS, a specific primary antibody raised in rabbit for TG-2 (Abcam, Cambridge, UK) diluted (1:100) with 2% goat serum was added and incubated further at 4° C. in a humid chamber overnight. After washing 3× with 1×PBS for 10 minutes, fluorescein-labeled goat anti-rabbit secondary antibody (1:200, Chemicon International, Temecula, Calif.) was applied and incubated for 90 minutes at room temperature. After washing and air-drying, slides were mounted with antifade medium containing DAPI (4,6-diamidino-2-phenylindole; Vectashield; Vector Laboratories, Burlingame, Calif.) to visualize the cell nuclei. Sections incubated with 2% goat serum were used as a control.

Fresh human and mouse scleral fibroblast cells were cultured on sterile chamber slides. Cells were washed with phosphate buffered saline (PBS) and fixed with ice-cold methanol:acetone (1:1) at −20° C. for 10 minutes and air-dried. Cells were permeabilised with 0.5% Triton-X 100 in PBS for 2 minutes at room temperature (RT). Non-specific sites were blocked with 1% bovine serum albumin (BSA), 0.3% Triton X-100 in PBS for 30 minutes at room temperature. The cells were then processed and stained as described above. A fluorescence microscope (Axioplan2; Carl Zeiss Meditec, GmbH, Oberkochen, Germany) was used to capture images. Procedures were repeated in triplicates from 3 different samples.

### Expression of TGases in the Ocular Surface and Associated Glands

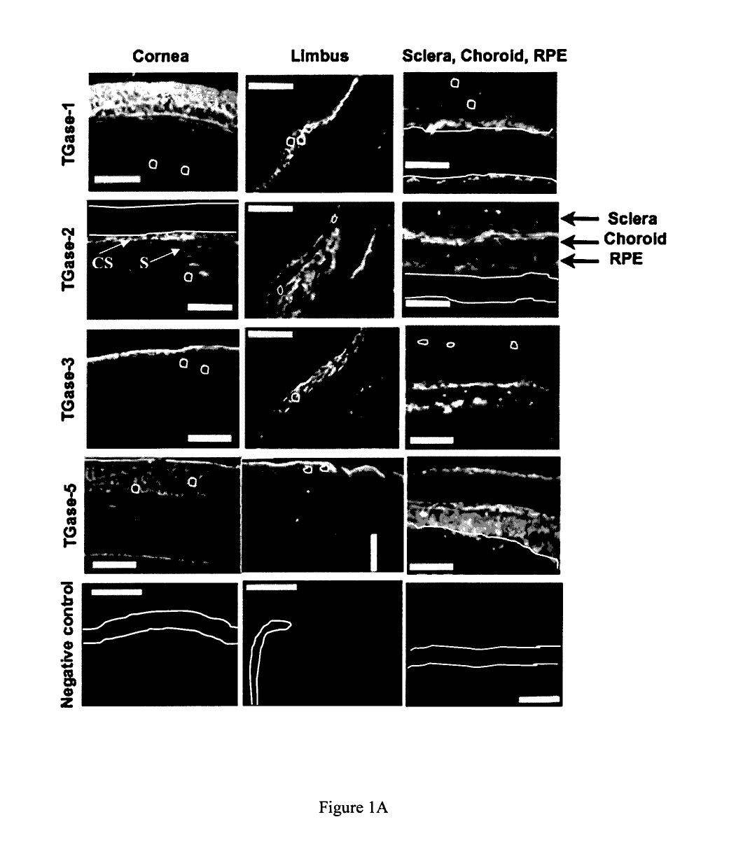

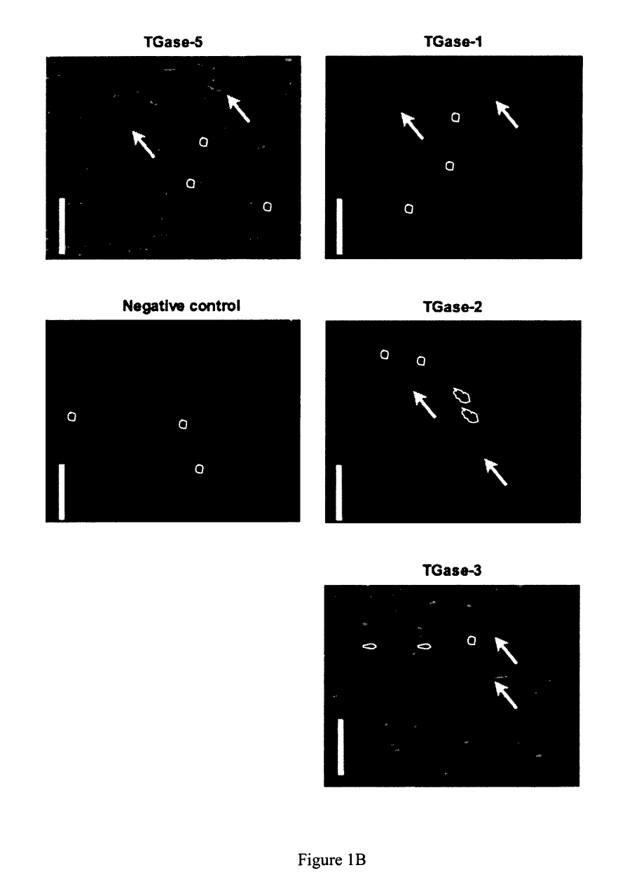

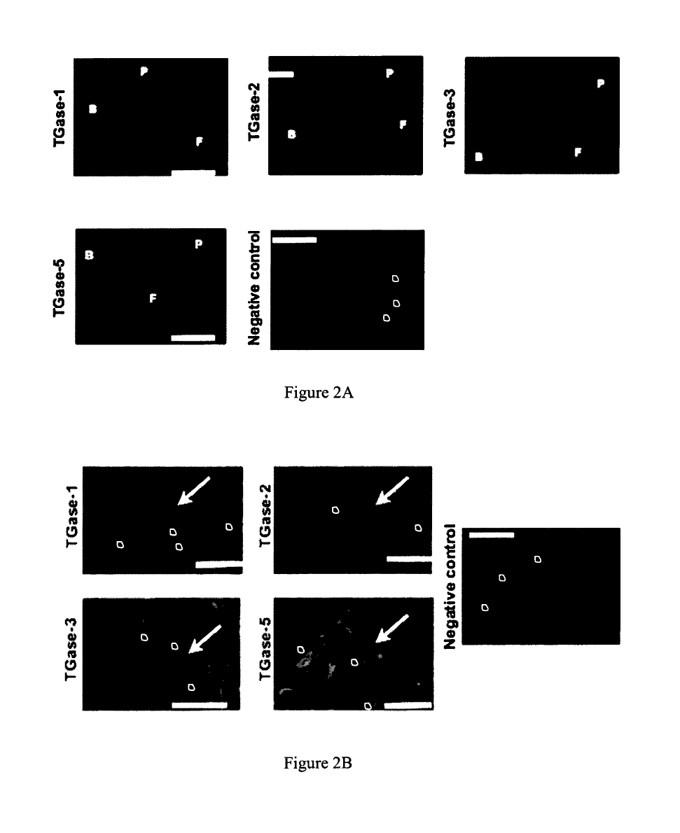

Positive immunostaining of TGs was shown in the mouse eye tissues (FIG. 1A) and human scleral tissues (FIG. 1B). TG-1, 3, 5 were localized in the entire mouse corneal epithelium, stroma and endothelium but TG-2 was present only in the corneal subepithelium and stroma. All TGs showed staining in the RPE, choroid and sclera, with scleral staining between the collagen fibre bundles. Sections used as the negative control were incubated with 2% goat serum without primary antibodies. Immunofluorescent staining showed the localization of TG-1, 2, 3 in the mouse palpebral, formiceal and bulbar conjunctiva but not TG-5 (FIG. 2A). All these TGs were expressed in mouse meibomian glands (FIG. 2B) but TG-2 was weakly detected.

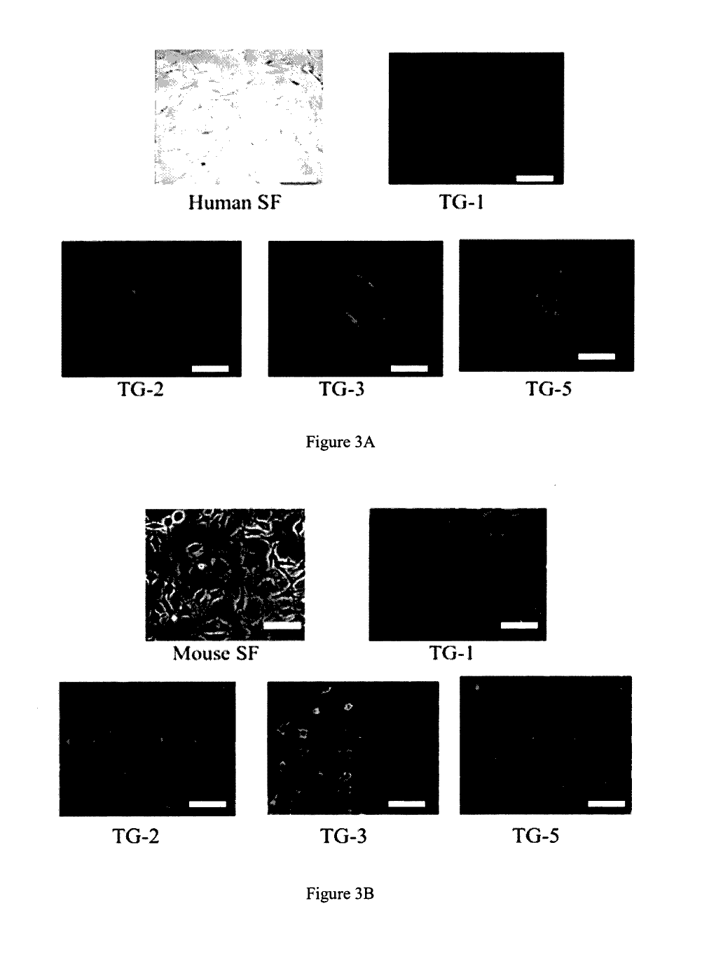

Cultured human and mouse scleral fibroblasts expressed all 4 TGs (FIG. 3). TG-1, 3 and 5 were located in the cytosolic and membrane compartments only whereas TG-2 was present in cell nucleus along with cytosolic and membrane compartment.

### Confirmation of TG Proteins in Mouse and Human Scleral Fibroblasts

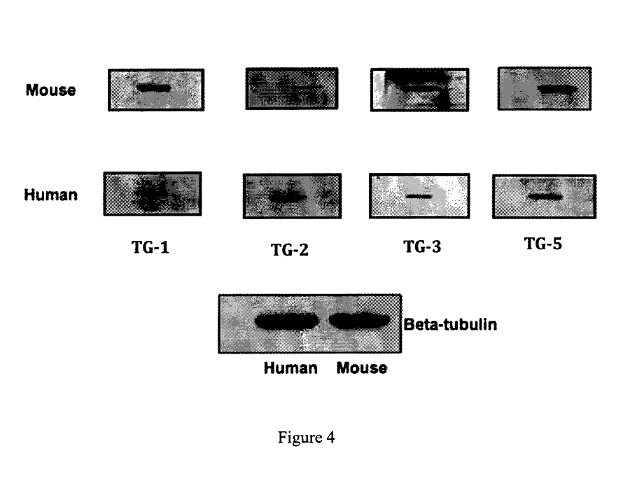

Western blot was carried out in the cultured mouse and human scleral fibroblast to confirm the expression of TGs at protein level and β-tubulin was used as a loading control (FIG. 4).

### Effect of TG Proteins Upon Muscarinic Agents Stimulation

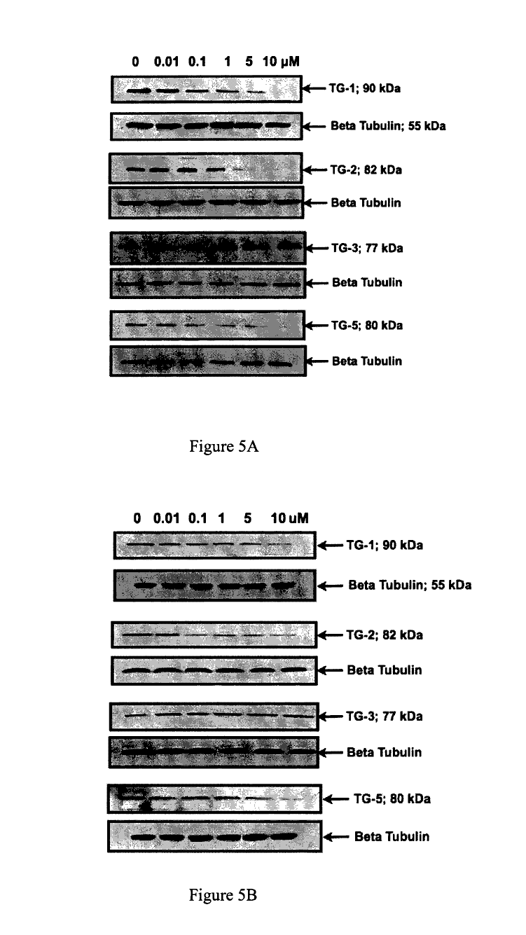

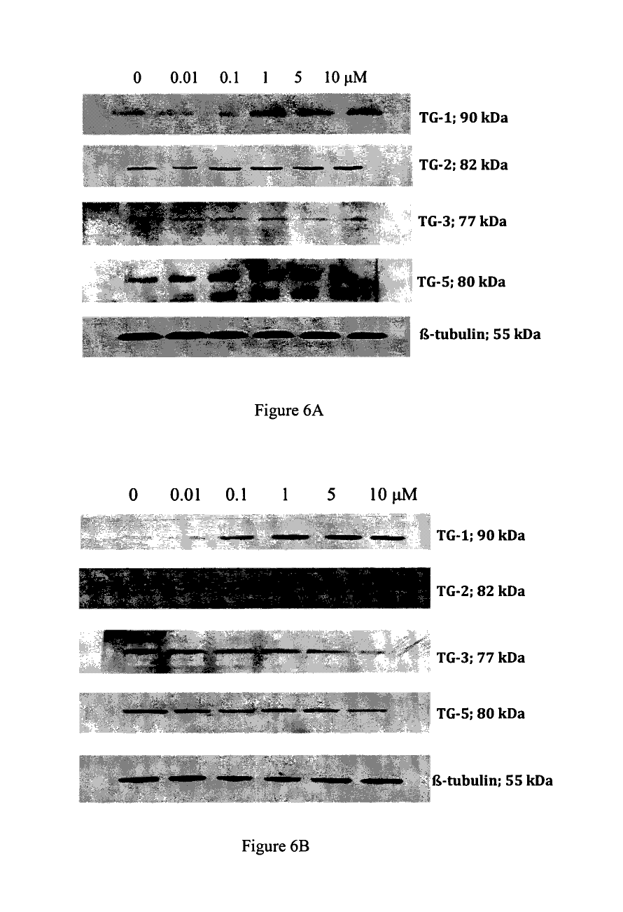

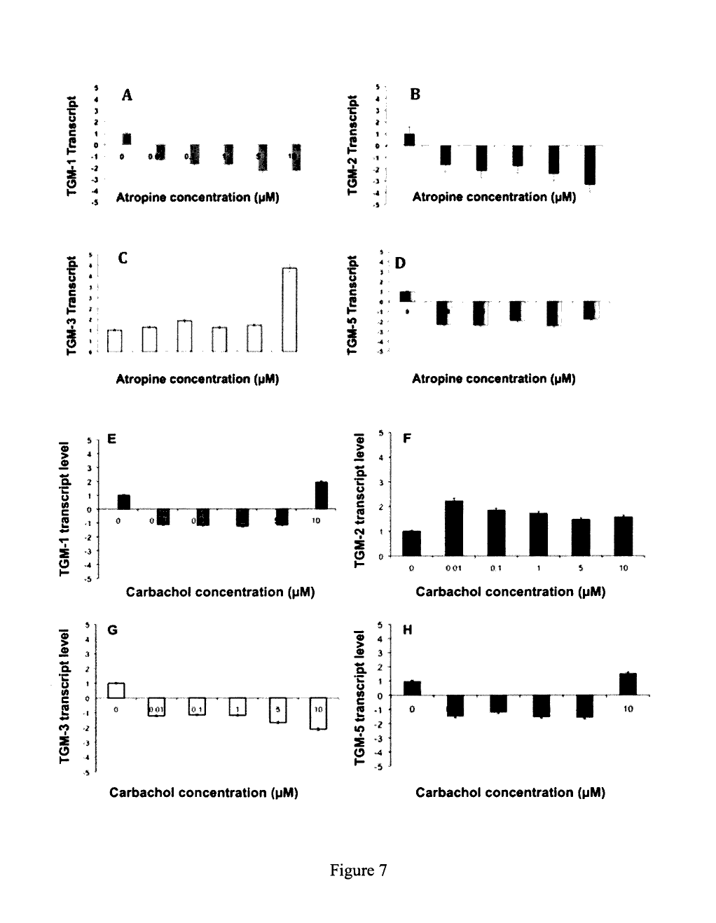

The mouse and human scleral fibroblasts were treated with atropine or carbachol at 0.01, 0.1, 1, 5 and 10 μM concentrations for 5 days. Following 5 days of treatment, the total cellular protein was extracted from these cells and TG proteins detected via Western blot analysis. After atropine treatment in both human and mouse scleral fibroblasts, the TG-1, 2 and 5 protein levels were reduced (FIGS. 5A and 5B respectively) and the converse was true with carbachol treatment (FIGS. 6A and 6B respectively). However, TG-3 protein level was increased in both human and mouse scleral fibroblasts after receiving atropine (FIGS. 5A and 5B respectively) and the converse observed with carbachol (FIGS. 6A and 6B respectively). These results were similar to changes in transcript levels (FIGS. 7A-H).

### Myopia and Sclera Connective Tissue

Despite the fact that there is more than one type of human myopia (late-onset and developmental) and animal myopia (lens induced, form deprivation, etc), it seems that all myopia is mediated by axial elongation resulting in a mismatch between the optical refractive properties of the anterior segment relative to the position of the photoreceptors on the retina. It has been reported that axial elongation could be mediated by alteration in the connective tissues of the sclera, a fibrous coat in the eye. Hence, without wishing to be bound by any theory, modulation of connective tissue molecules in the sclera may represent a strategy for arresting all types of myopia development, regardless of the initiating stimulus.

### Example 5

### Scleral Fibroblast Cell Proliferation Assay

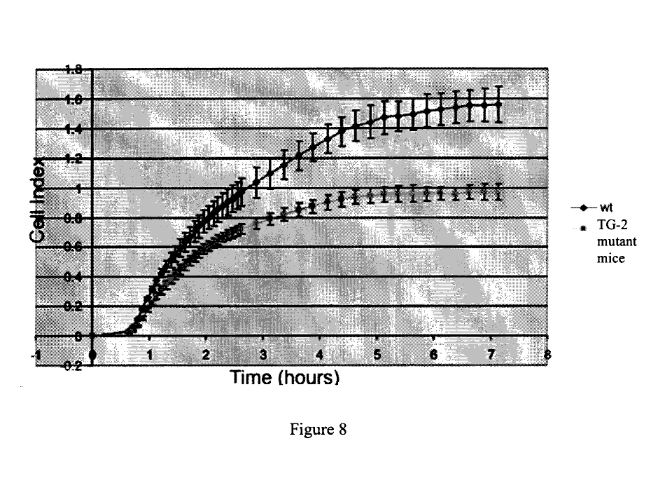

Roche XCELLigence system RTCA SP (Roche Applied Science, IN) was used for the monitoring of mouse scleral fibroblast (SF) proliferation in real time. Cells were seeded in microtiter plates containing microelectronic sensors (96× E-Plate). The interaction of cells with the electronic biosensors generates a cell-electrode impedance response that is expressed as cell index allows for cell numbers to be detected. Cells that had previously been transfected were trypsinised and counted. 5000 TG-2 mutant and WT SF cells were seeded in 100 μl of media in duplicates in a 96× E-Plate, proliferation was monitored in real time. Cell-sensor impedance was measured every 5 minutes for the first 2 hours, every 15 minutes for the following 7 hours. The results of this assay are shown in FIG. 8. In SFs derived from TG-2 mutant mice cell growth was significantly reduced after 7 hours and continued to be further reduced over time compared to WT SF cell growth.

### Example 6

### Electrophoresis and Immunoblotting

Human and, mouse scleral fibroblast proteins in the supernatant were separated by SDS-PAGE, transferred to nitrocellulose membranes, blocked in 5% BSA in TBST (10 mM Tris-HCl [pH 8.0], 150 mM NaCl, and 0.0.05% Tween-20) for 2 hours at room temperature, and incubated with the same anti-TG-2 antibody described herein at a dilution of 1:1000 and anti β-tubulin antibodies used as a positive control, for 1 hour at room temperature. The membranes were washed 3 times in TBST and incubated with HRP-conjugated secondary antibody (Chemicon International) at a dilution of 1:2500 for 1 hour at room temperature. Immunoreactive bands were visualized using the enhanced chemiluminescence method (GE Healthcare, Buckinghamshire, UK). The membrane was wrapped in plastic and placed against an X-ray film to expose for an appropriate length of time (30 seconds-5 minutes).

### Muscarinic Receptors in Myopia Development

Little is known to which receptors are responsible for the blocking growth of axial length. Pirenzepine has been tested in children as well, and it is not as effective as atropine. However, pirenzepine is somewhat more selective for the ml receptor. Other specific muscarinic receptors were studied to arrest myopia progression in animal models. As there are few useful or selective mAChR blocking agents, the only way to determine which receptors are needed is to use mice with selective muscarinic receptor knockouts.

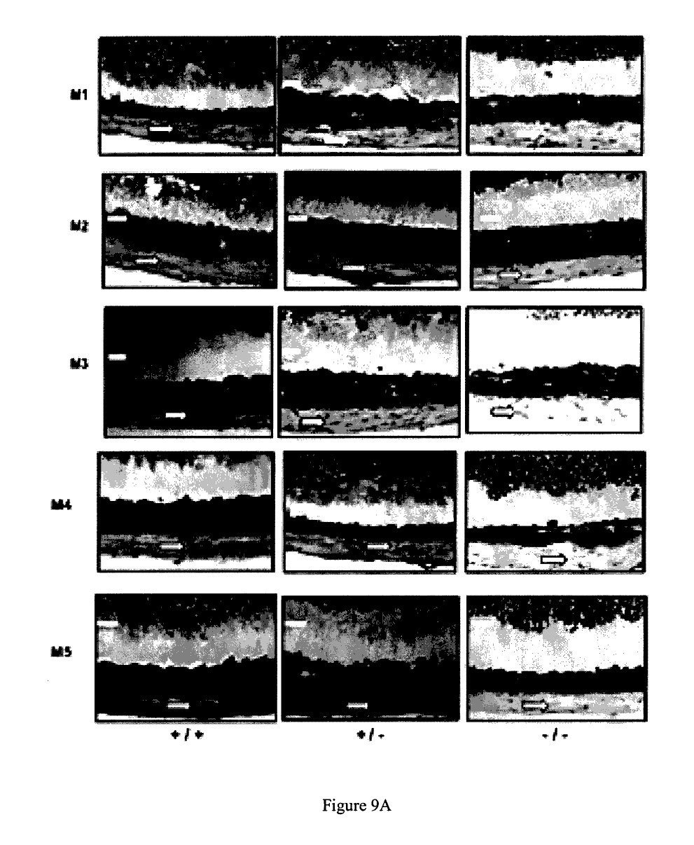

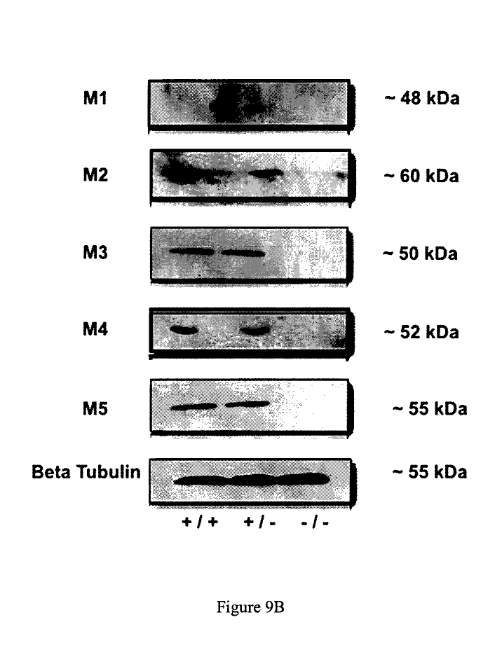

Immunohistochemistry and Western blot showed the expression of all five muscarinic receptor subtypes in sclera from wild type (wt) mice and not expressed in the mutant mice (FIGS. 9A and 9B). Using mAChR mutant mice for experimental myopia, M1^{-/-}, M4^{-/-} and M5^{-/-} axial growth was significantly 250 μm longer than the M2^{-/-} and 300 μm longer than the M3^{-/-}. In M1^{+/+}, M2^{+/+}, M3^{+/+}, Me^{+/+} and M5^{+/+} mice axial growth was not significantly different. Without wishing to be bound to any theory, these findings provide initial evidence that in terms of scleral growth in experimental myopia M1, M4 and M5 muscarinic receptors contribute more than M2 and M3. M2 and M3 mutant mice were remained hyperopic at 6 weeks after induction as compared to M1, M4, M5 mutant and wild type mice, they had a myopic shift even after 4 weeks of induction.

### Transglutaminases and Muscarinic Receptors

There is much literature in animal and human myopia to support the role of mAChRs, especially the clinical trials concerning the efficacy of the muscarinic antagonists atropine and pirenzepine, in retarding childhood myopia.

Anti-muscarinic drugs were able to change collagen and other structural molecules in animal models of myopia, establishing a link to scleral connective tissue in these pathways. However as mentioned above, even though muscarinic receptor subtypes may be involved in scleral remodeling, their exact downstream molecules are not known.

### Example 7

### TG-2 Colorimetric Microassay

This assay was designed to assess the cross-linking activity of TG-2 between two proteins. TG activity was measured with TG-2 colorimetric Microassay kit (TG-Covtest, Covalab, Cambridge, UK). 150-200 μg of total protein was used per well for the TG-2 assay. Transglutaminase standards (Sigma) were used as a positive control. EDTA was used to inhibit the enzyme activity as a negative control.

The approximate volume of reagents to use, the wavelengths to use to read absorbance, the incubation times were determined from a previous study that have carried out experiments with similar principles as described in Perez Alea M, Kitamura M, Martin G, Thomas V, Hitomi K, El Alaoui S., “Development of an isoenzyme-specific colorimetric assay for tissue transglutaminase 2 cross-linking activity”, Anal Biochem. 2009 Jun. 15; 389(2):150-6, Epub 2009 March 24. All reagents used and protein coated plates were part of the kit. Biotin cadaverine was reconstituted with 6 ml of deionized water and 90 μl was added into each well of the 96 well plate that comes coated with CBZ-GLN-GLY. The rest of the steps were followed as in the manufacturer's instructions and finally absorbance was read at 450 nm using a microplate reader (TECAN, Austria). All incubations were performed with gentle shaking on a laboratory orbital shaker.

### Transglutaminase-2 Activity

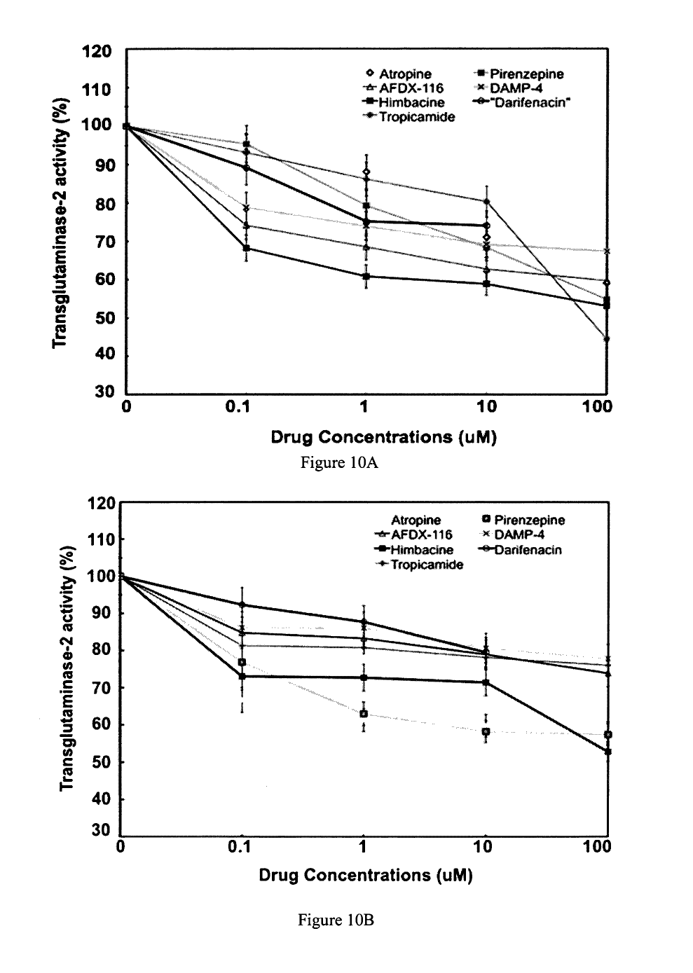

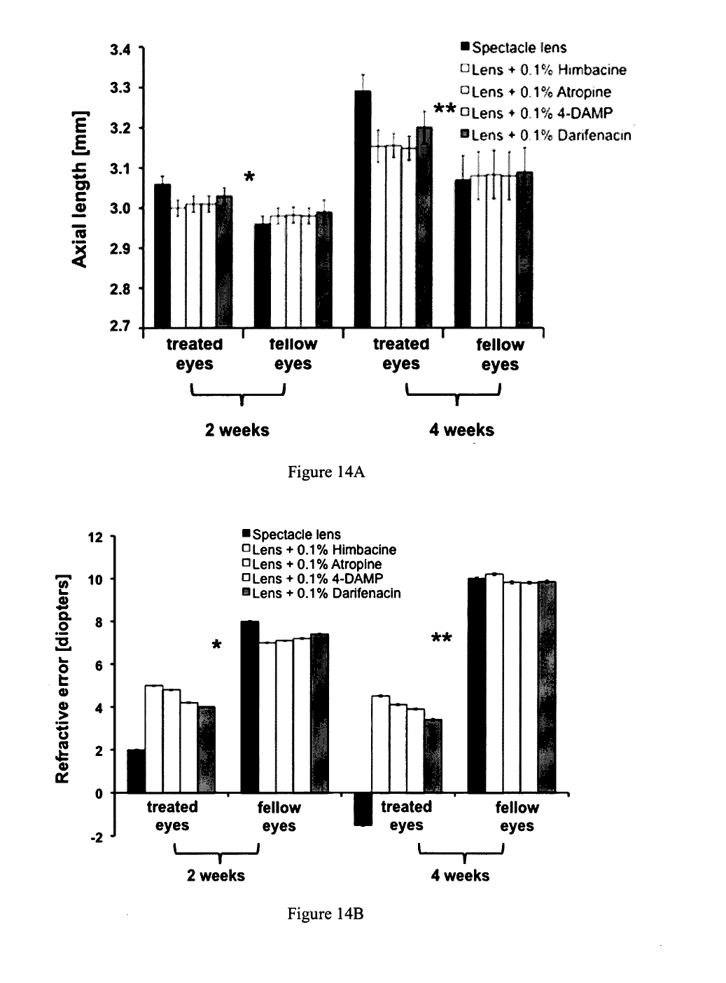

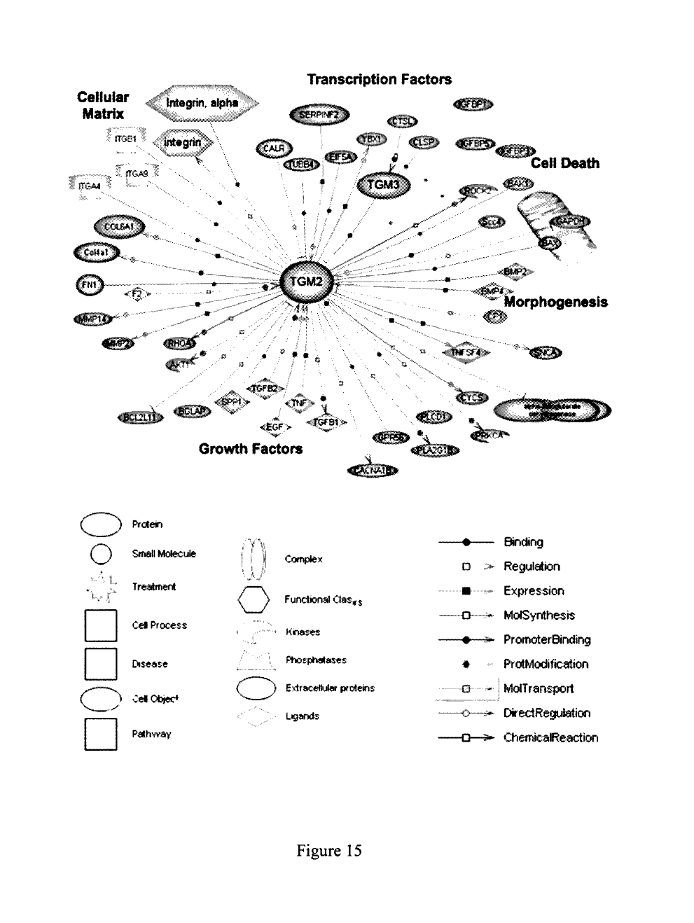

The TG-2 colorimetric assay serves as a high throughput and quantitative assay to assess transamidase activity. The mouse and human scleral fibroblasts treated with atropine, pirenzepine, AFDX-116, himbacine, 4-DAMP, darifenacin and tropicamide at baseline, 0.1, 1, 10 and 100 μM for 5 days. The transamidase activity of endogenous cellular TG-2 activity was reduced by antagonists' treatment in a concentration-dependent manner in human and mouse scleral fibroblasts (FIGS. 10A and 10B respectively). Moreover TG-2 activity was most significantly reduced with himbacine treatment in both cells and also with darifenacin at low concentrations, higher concentrations was kept constant for toxicity effect. In vivo studies with these drugs were also conducted to determine the effect on myopia development (see FIGS. 14 and 15).

### Transglutaminase and Connective Tissue

TG-2, a multi-functional tissue enzyme of the transglutaminase family, plays a central role in wound healing, apoptosis and extra-cellular matrix (ECM) production. There are a few ways that TG-2 may be involved in remodeling of ECM. First, TG-2 may affect the covalent cross-linking of ECM molecules in the extracellular space, hence affecting stabilization and degradation of these molecules. Secondly, TG-2 may affect the motility, adhesion and survival of the ECM producing fibroblasts, hence influencing the amount of connective tissue molecules produced.

In the sclera, a previous study indicated that TG activity was localized to mainly in the episcleral vessel walls. In various studies, TG-2 has also been implicated in the signaling pathways of extracellular matrix molecules such as fibronectin and collagen subtypes. In the cornea epithelium for example, it is involved in the regulation of cornified envelope proteins and apoptosis.

### Example 8

### Refractive Error and Axial Length Assessment

Each eye was refracted every week to measure the refractive error using automated infrared photorefraction as described in Barathi, V. A., Beuerman, R. W., Schaeffel, F., 2009a, “Effects of unilateral topical atropine on binocular pupil responses and eye growth in mice”, Vision Res 49, 383-387. By using Optical Low Coherence Interferometry (OLCI, AC Master, Carl-Zeiss), the axial length of the eye was measured in-vivo as described in Yin G C, Gentle A, McBrien N A, “Muscarinic antagonist control of myopia: a molecular search for the M1 receptor in chick”, Mol. Vis. 2004; 13(10):787-93 and Barathi, V. A., Boopathi, V. G., Yap, E. P., Beuerman, R. W., 2008, “Two models of experimental myopia in the mouse”, Vision Res 48, 904-916. This method has been demonstrated to provide improved resolution and reproducibility, and allowed serial monitoring of axial length of the eyeball in various stages of myopic induction. Differences of refractive power and axial length between eyes were calculated. Independent t-tests were used to compare experimental and control eyes in wild type and TGM2-deleted or M₁-M₅ mutant mice.

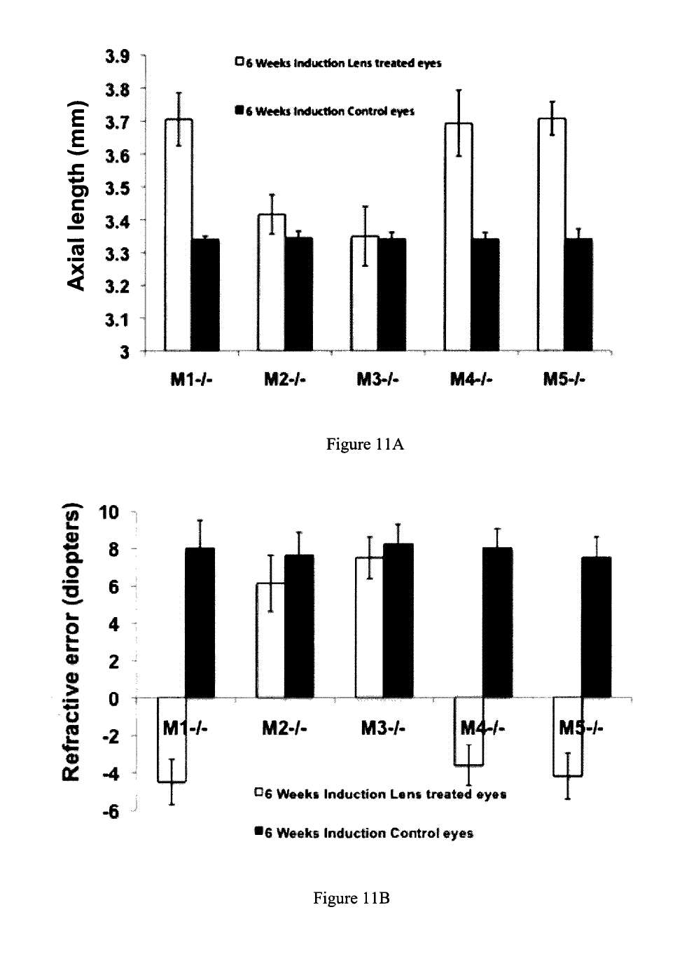

Axial length (mm) and refractive error (diopters) of the mouse eye was presented in FIGS. 11A and 11B respectively. These results confirmed that mAChR 2 and 3 mediates the myopia formation in mouse.

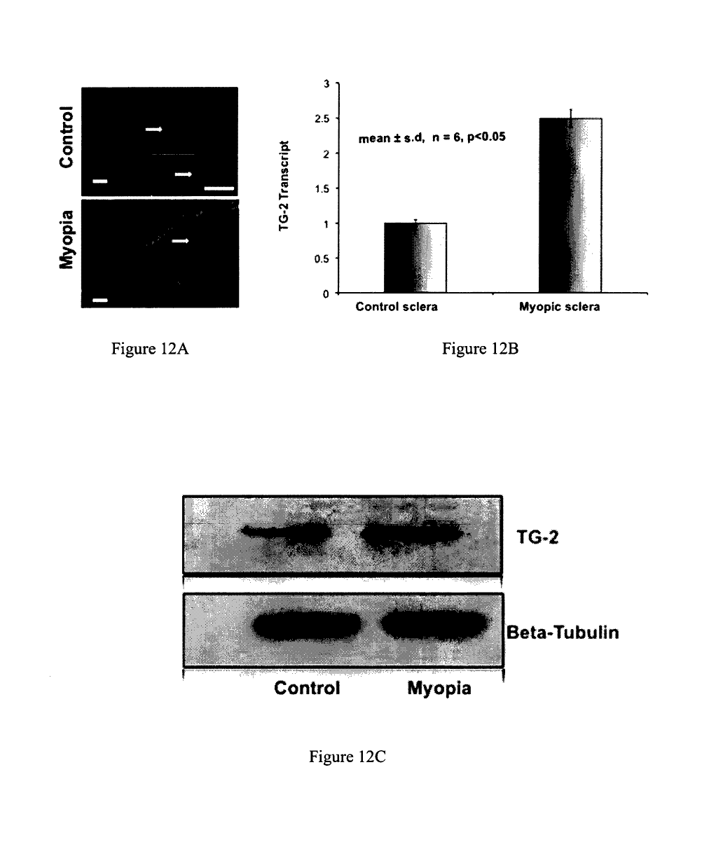

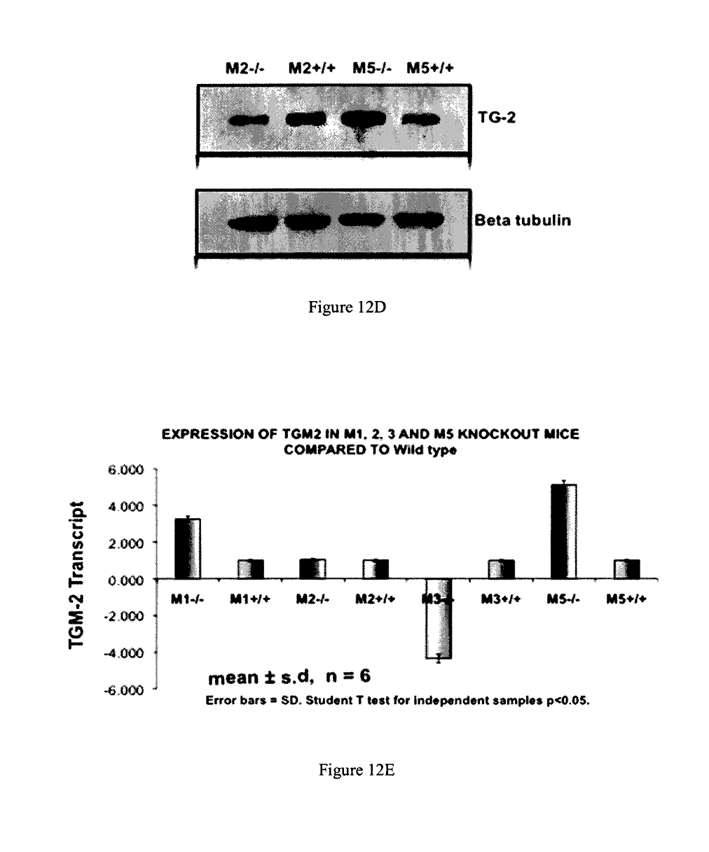

Without wishing to be bound by any theory, it is shown that regulation of TG-2 is necessary for the growth of the sclera in experimental myopia (FIGS. 12A-C). In this context, TG-2 protein and transcript were reduced in sclera tissue in m2 mutant murine eyes compared to m5 mutant murine eyes (FIGS. 12D and E). These results suggest that TGM-2, M₂ and M₃ mediate the effects of myopia in mouse model.

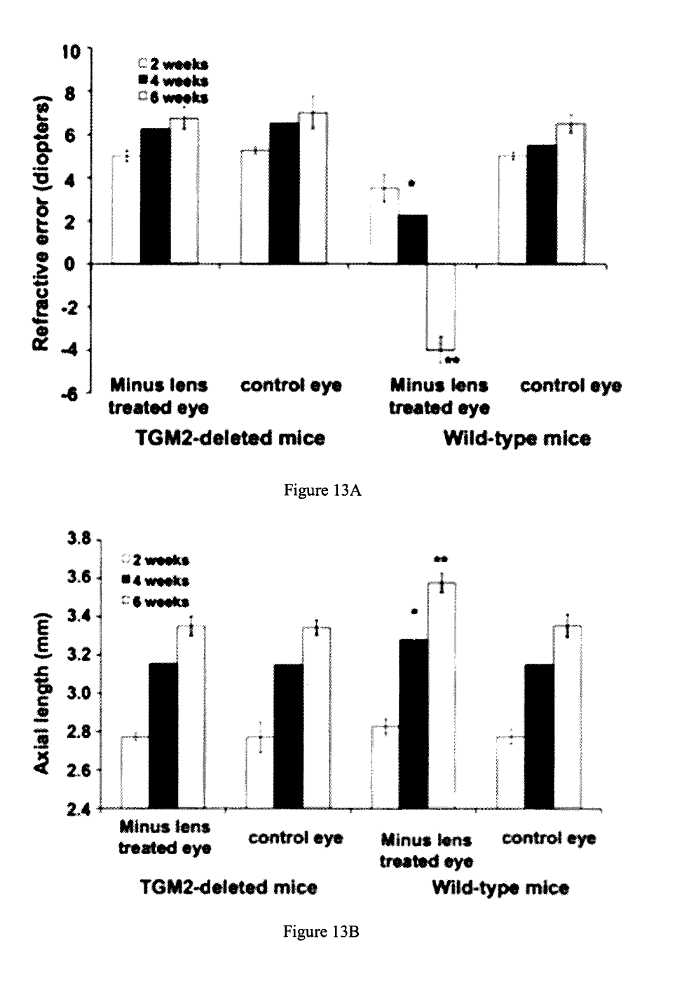

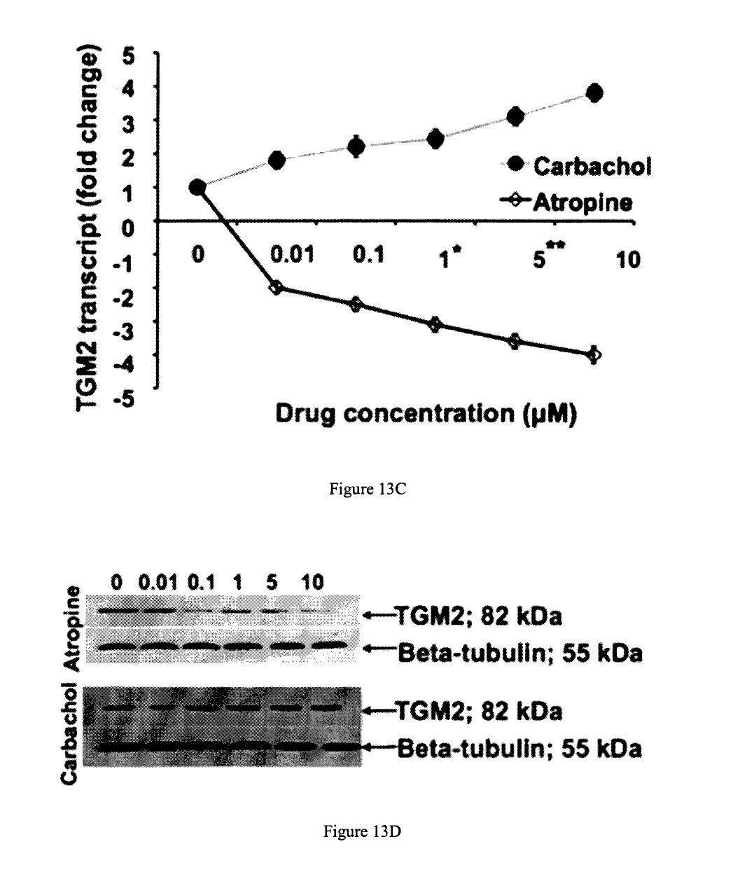

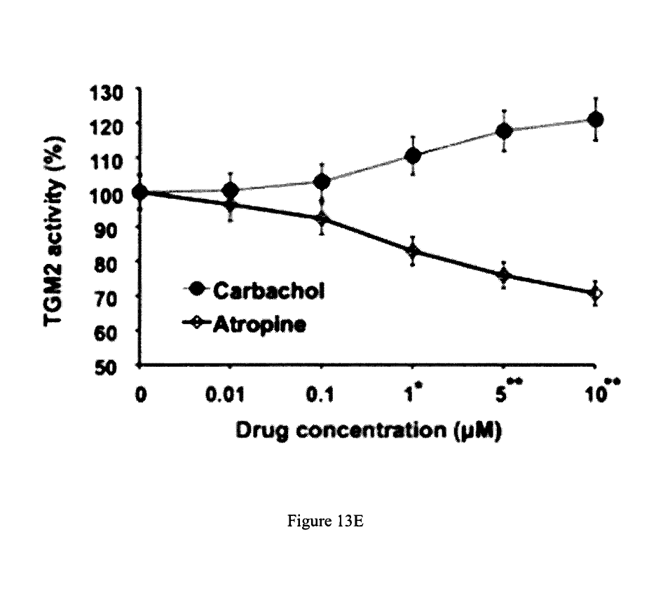

Upon myopia induction with negative lens, homozygous TGM2-deleted mice were found to remain hyperopic at week 8 (6 weeks after induction) compared to wild type mice (FIG. 13A). The same effect was found with the diffuser treatment in both TGM2-deleted mice and wild type mice (data not shown). Plano powered lens did not induce myopia in wild type mice (data not shown). Axial length elongation was significantly higher in negative lens treated wild type mice at week 8 (6 weeks after induction), but not in TGM2-deleted mice (FIG. 13B), compared to contra-lateral uncovered eyes of the same animals. When the study was repeated with diffuser lenses (which prohibited sharply focused retinal images) in place of the negative lenses, similar results were obtained (data not shown). Therefore, the present results show that M₂, M₃ and TGM-2 have a critical role in the formation of myopia in this animal model.

Both muscarinic receptors and TGM-2 signals through GTPase, and both can increase calcium, muscarinic receptors acting on ion channels and TGM-2 acting via phospholipase C. In sclera-derived fibroblasts from wild-type mice, treatment with pan-muscarinic antagonist atropine and specific antagonist induced a reduction in the level of TGM-2 transcript (FIG. 13C), and in protein (FIG. 13D, top and middle) and cross-linking activity (FIG. 13D bottom), whereas the pan-muscarinic agonist carbachol had the opposite effects (FIG. 13D).

Mice eyes were treated with different muscarinic antagonists' to determine the effective drug for the reduction of myopia progression. Mouse eyes treated with −15D spectacle lens, lens with 0.1% himbacine, lens with 0.1% darifenacin, lens with 0.1% 4-DAMP and lens with 0.1% atropine for 2 weeks and 4 weeks (n=6 mice in each group). Right eyes were experimental and left eyes served as contra-lateral control. In vivo axial length was measured at 2 weeks and 4 weeks after treatment. The axial length was significantly reduced in the drug treated eyes as compared to minus lens treated eyes (FIG. 14A). Among all the drugs, himbacine showed the most significant amount of reduction in the mouse myopia progression. Automated infrared photorefractor was used to measure the refractive error measurements. The refractive error was shifted from myopic to hyperoic after receiving the drugs (FIG. 14B).

### Transglutaminase and Myopia

In the mouse model of myopia induced by uni-ocular negative lens wear as described herein, TG-2 protein and TGM-2 RNA levels were found to be elevated in myopic eye, compared to the control eye. In fibroblasts cultured from scleral explants from these animals, treatment with atropine, a pan muscarinic receptor antagonist induced a reduction in the level of TG-2 protein and RNA. Specific muscarinic antagonists such as himbacine, 4-DAMP, darifenacin, pirenzipine, AFDX-116 and tropicamide were also tested the mouse and human scleral fibroblasts. The TG-2 protein level was significantly reduced with himbacine treatment in both mouse and human scleral fibroblasts. The effect was also significant with very low concentrations of 4-DAMP and darifenacin treatment.

Cell adhesion and cell spreading are integral functions regulated by TGs. Primary fibroblasts from TGM-2 mutant mice have decreased adherence to culture vessels. Cell-matrix interactions are critical for spreading and migration of cells, as well as the organization of extracellular matrix.

TGM-2 deficient fibroblasts can still attach to extracellular matrix, but shows deficient spreading and migration, and show defects in turnover of focal adhesion and formation of stress fibers. These defects in motility and adhesion kinase phosphorylation are un-related to the transamidase function but are instead related to the G protein function of TG-2. TG-2 is also required for the membrane type 1-MMP dependent activation of MMP-2. Crosslinking by TG-2, if perturbed, results in reduced collagen matrix contraction and activation of gelatinase. Hence both the G protein and transamidase functions of TG-2 independently contribute to wound healing or ECM remodeling processes. Without wishing to be bound by any theory, if TG-2 acts on scleral fibroblasts in the same way as in fibroblasts from other parts of the body then there is a strong basis for its action on scleral ECM remodeling and hence myopia formation.

### Example 9

### Real-Time Comparative PCR

Total RNA was isolated from cultured human and mouse scleral fibroblasts using TRIzol reagent (Invitrogen life technologies, USA) in accordance with the manufacturer's instructions Chomczynski P, Sacchi N., “Single-step method of RNA isolation by acid guanidinium thiocyanate-phenol-chloroform extraction”, Anal. Biochem. 1987 April; 162(1):156-9. Genomic DNA was removed by digestion with DNase I (Amp Grade; Invitrogen-Gibco) for 15 minutes at room temperature. One microgram of total RNA was reverse-transcribed with random hexamers by using a first-strand cDNA synthesis kit (Invitrogen-Gibco). qPCR was performed in a 384-well plate format on an Roche 480 LightCycler Detection System (Roche Applied Science, Mannheim, Germany) with efficiency corrected software 4.0. PCR was performed using 50 ng of cDNA of each sample. The pre-validated hydrolysis probes for TG-2 were from human and mouse universal probe library (Roche, CA) and the primers for human and mouse are shown in Table 3. GAPDH Internal Standard (Roche) was used as an endogenous control. To standardize and evaluate scleral gene expression, aliquots of the same cDNA (50 ng) preparation were used as templates in all PCR reactions. The data was analyzed by comparative C_{T} (MCT) method as previously described in Brink N, Szamel M, Young A R, Wittern K P, Bergemann J., “Comparative quantification of IL-1, IL-10, Il-10r, TNF and IL-7 mRNA levels in UV-irradiated human skin in vivo”, Inflamm Res. 2000 June; 49(6):290-6.

TABLE 3
Human and Mouse TGs probes and primers for qPCR reactions

| | | | Prevalidated |
|---|---|---|---|
| Gene | Primers | Sizes | Probes |
| hTGM 2 | 5′ AGG GTG ACA AGA GCG AGA TG-3′ (Fw) (SEQ ID NO: 1) | 96 bp | #32 |
| | 5′ TGG TCA TTC ACG ACT CCA C-3′ (Rev) (SEQ IDNO: 2) | | |
| mTGM 1 | 5′ GCC CTT GAG CTC CTC ATT G 3′ (Fw) (SEQ ID NO: 3) | 74 bp | #10 |
| | 5′ CCC TTA CCC ACT GGG ATG AT-3′ (Rev) (SEQ ID NO: 4) | | |
| mTGM 2 | 5′ GGT GAT CCT CGC TTG AGT GT-3′ (Fw) (SEQ ID NO: 5) | 94 bp | #17 |
| | 5′ CTC CAA ATC ACA CCT CTC CAG-3′ (Rev) (SEQ ID NO: 6) | | |
| mTGM 3 | 5′ GAT CAC AGC TGT TTG CAA GG-3′ (Fw) (SEQ ID NO: 7) | 107 bp | #31 |
| | 5′ CAT GAG CCT GTT CCA GCA C-3′ (Rev) (SEQ ID NO: 8) | | |
| mTGM 5 | 5′ TCC ATC CAG CTG TCT GTG G-3′ (Fw) (SEQ ID NO: 9) | 91 bp | #26 |
| | 5′ AGG GCC ACC TCT AGT CCT GT-3′ (Rev) (SEQ ID NO: 10) | | |

### Example 10

### RNA Interference Assay

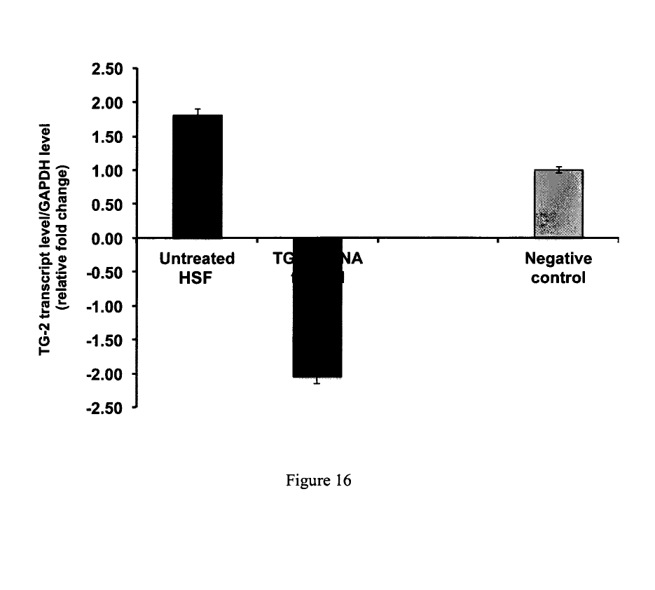

Human scleral fibroblasts (HSF) of Passage 4 were used. 3.5×10⁴ HSF cells were plated in a 12 well plates and maintained in DMEM supplemented with 10 percent FBS and 1% penicillin and 1% streptomycin at 37° C., 5% CO₂ prior to transfection. To block the function of TG-2 in HSF cells, small interfering (si)RNA molecules targeted at TG-2 mRNA were used. All siRNA used were purchased from Qiagen. Transfection was done following manufacturer's instructions. Briefly, 30 nM of scrambled siRNA-negative control and TG-2 siRNA (NM_{—}004613) were diluted in basal DMEM and incubated with HiPerfect transfecting reagent (Qiagen) at room temperature for 10 minutes before adding dropwise to cells. Cells were then placed back into incubator and monitored for the following 48 hours. Knockdown efficiency, which refers to the percentage of reduction in mRNA/protein of the targeted gene, was determined using q-PCR to confirm that gene of interest has achieved a knockdown efficiency of 60 percent for siRNA against TG-2 in human scleral fibroblasts (HSF) compared against untreated HSFs and the negative control. The results of this assay are shown in FIG. 16.

### Example 11

### Monitoring of Cell Growth in Real Time

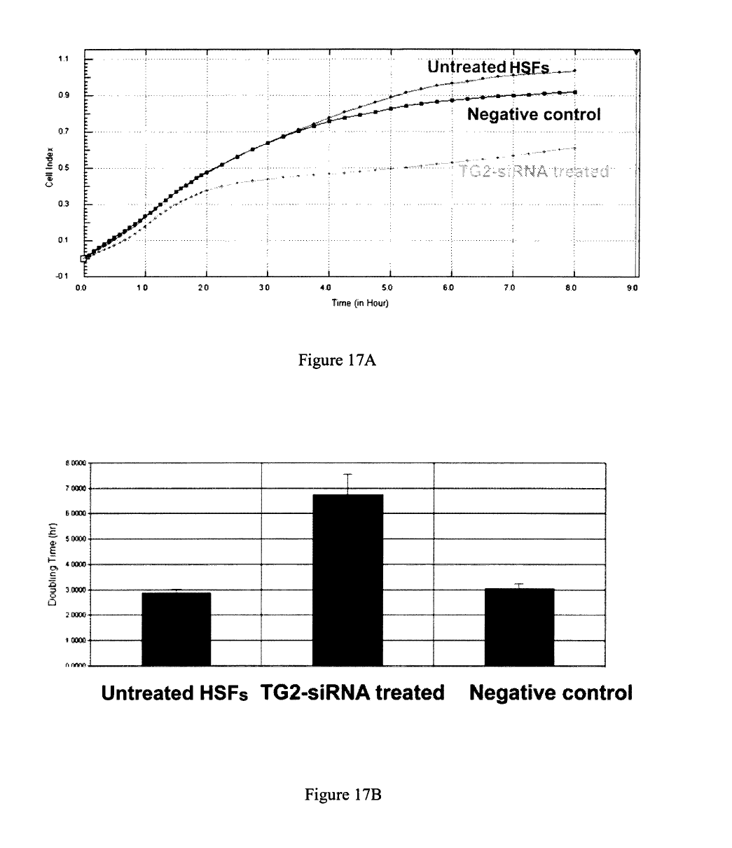

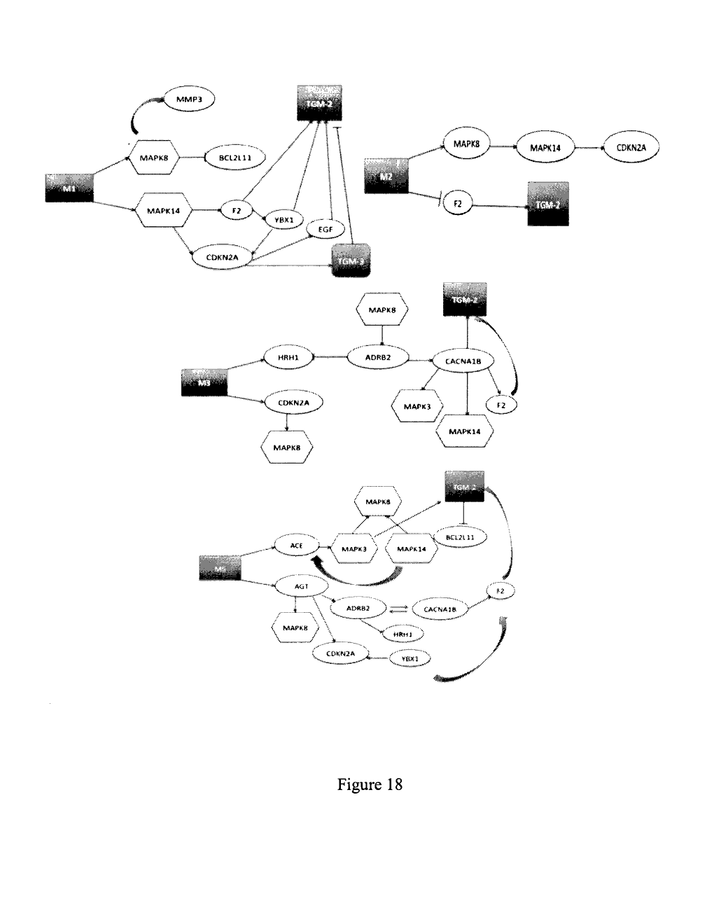

Cell growth and proliferation were assessed in real time using impedance technology via XCELLigence system RTCA SP (Roche Applied Science, IN). The interaction of cells with the electronic biosensors generates a cell-electrode impedance response that is expressed as cell index; allowing cell numbers to be detected. 2000 HSF cells transfected with various siRNAs and an untreated control were seeded in 100 μl of media in quadruplicates in a 96× E-Plate. Cell-sensor impedance was measured every 5 minutes for the first 2 hours, every 15 minutes for the following 7 hours and every 1 hour for the rest of the process. An increase in cell index correlates with increase in cell number. The results of the assay are shown in FIG. 17A.

Doubling time, which is the time required to achieve double population, was also observed to be significantly higher (at least 2×) for TG-2-siRNA treated HSF as compared to untreated HSFs and/or the negative control (FIG. 17B).

### Example 12

To test the effect of TG-2 inhibitors on cultured scleral fibroblast growth, cultured SF cells at passage 4 were treated with TG-2 inhibitors at baseline, 0.1, 1, 10 and 100 μM for 5 days. The SF cell growth was monitored by XCELLigence cell impedance.

### Effect of TG-2 Inhibitors on Cultured Scleral Fibroblast Growth

D003[1,3-Dimethyl-2-((2-oxopropylthio)imidazolium chloride] and R002 (TAMRA-DON) inhibitor (more specific to TG-2) treated SF cell growth and cell numbers were significantly reduced at 0.1 μM and this was further reduced with concentration dependent manner (n=3, ANOVA, p<0.01). No change was determined in the cell morphology. However, cells treated with MDC (Monodansyl caderverine; TG competitive inhibitor) were only effective at 10 μM for human and 50 μM for mouse SF. The higher concentrations caused cell death and morphological change.

The listing or discussion of a previously published document in this specification should not necessarily be taken as an acknowledgement that the document is part of the state of the art or is common general knowledge. All documents listed are hereby incorporated herein by reference in their entirety.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognise that the invention is also thereby described in terms of any individual member or subgroup of members of the Markush group.

1.-25. (canceled)

26. A method of treating myopia, comprising administering to a subject a TG-2 inhibitor, wherein the TG-2 inhibitor is selected from the group consisting of darifenacin or an analogue thereof; 1,1-Dimethyl-4-diphenylacetoxypiperidinium iodide (4-DAMP) or an analogue thereof; a nucleic acid molecule that inhibits expression of transglutaminase-2 (TGM-2); and an analogue of himbacine;
wherein the analogue of himbacine is a compound of Formula I: 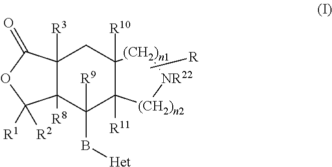
or a pharmaceutically acceptable salt thereof, wherein:
R is 1 to 3 substituents independently selected from the group consisting of H, C₁-C₆ alkyl, halogen, hydroxy, amino, (C₁-C₆)alkyl-amino, (C₁-C₆)-dialkylamino, (C₁-C₆)alkoxy, —COR¹⁶, —COOR¹⁷, —SOR¹⁶, —SO₂R¹⁶, —SO₂NR¹⁷R¹⁸, —NR¹⁷SO₂R₁₈, NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, C₃-C₆cycloalkyl, aryl(C₁-C₆)alkyl, hydroxy(C₁-C₆)alkyl, amino-(C₁-C₆)-alkyl, aryl and thio(C₁-C₆)alkyl;
R¹ and R² are independently selected from the group consisting of H, C₁-C₆ alkyl, fluoro(C₁-C₆)alkyl, difluoro(C₁-C₆)alkyl, trifluoro-(C₁-C₆)alkyl, C₃-C₆cycloalkyl, C₂-C₆ alkenyl, aryl(C₁-C₆)alkyl, hydroxy-(C₁-C₆)alkyl, amino(C₁-C₆)alkyl, aryl and thio(C₁-C₆)alkyl; or and R¹ and R² together form an ═O group;
R³ is H, hydroxy, C₁-C₆alkoxy, aryloxy, aryl(C₁-C₆)alkyloxy, heteroaryloxy, heteroaryl(C₁-C₆)alkyloxy, (C₃-C₆)cycloalkyloxy, —SOR¹⁶, —SO₂R¹⁷, —SO₂NR¹⁸R¹⁹, —SR¹⁸, —SO₃H, —C(O)OR¹⁷, —C(O)NR¹⁸R¹⁹, —OC(O)R³², —OC(O)NR³³R³⁴, —(CR³³R³⁴)ₙOR³², —NR⁴R⁵, —NR³³COOR³², —NR³³COR³², —NR³³S(O)₂R³², —NR³³CONR³³R³⁴, —NR³³S(O)₂NR³³R³⁴, —(CR³³R³⁴)ₙNR⁴R⁵, —(CR³³R³⁴)ₙNR³³COR³², —(CR³³R³⁴)ₙNR³³COR³², —(CR³³R³⁴)ₙNR³³S(O)₂R³², —(CR³³R³⁴)ₙNR³³CONR³³R³⁴, —(CR³³R³⁴)ₙNR³³S(O)₂NR³³R³⁴, (C₁-C₆)alkyl, halogen, C₃-C₆cycloalkyl, C₂-C₆alkenyl, —CN, aryl, heteroaryl, heterocycloalkyl, —P(O)(OR⁷)₂ or (C₁-C₆)alkyl substituted by 1 to 3 substituents independently selected from the group consisting of halogen, —OH, —NH₂, aryl, —COOH, —SO₃H, thio and (C₁-C₆)alkylthio;
n is 1, 2, 3 or 4;
n1 and n2 are independently 0-3, provided both are not 0;
Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of C₁-C₆alkyl; —NR⁴R⁵; —NHCOR²⁶; —NHSO₂R¹⁶; R²¹-aryl; aryl wherein adjacent carbons form a ring with a methylenedioxy group; and R²¹-heteroaryl;
R⁴ and R⁵ are independently selected from the group consisting of H, C₁-C₆ alkyl, phenyl, benzyl and C₃-C₆ cycloalkyl, or R⁴ and R⁵ together are —(CH₂)₃—, —(CH₂)₄—, —(CH₂)₅— or —(CH₂)₂NR⁷—(CH₂)₂— and form a ring with the nitrogen to which they are attached;
R⁷ is H or (C₁-C₆)alkyl;
R⁸, R¹⁰ and R¹¹ are independently selected from the group consisting of R¹ and —OR¹;
R⁹ is H, OH, —NR⁴R⁵, C₁-C₆alkoxy, halogen or halo(C₁-C₆)alkyl;
B is —(CH₂)n₃- or cis or trans —(CH₂)n₄CR¹²═CR^{12a}(CH₂)n₅, wherein n₃ is 0-5, n₄ and n₅ are independently 0-2, and R¹² and R^{12a} are independently selected from the group consisting of H, C₁-C₆ alkyl and halogen;
R¹⁶ and R^{16a} are independently selected from the group consisting of C₁-C₆ alkyl, phenyl and benzyl;
R¹⁷, R¹⁸ and R¹⁹ are independently selected from the group consisting of H, C₁-C₆alkyl, phenyl and benzyl;
R²¹ is 1 to 3 substituents independently selected from the group consisting of H, —CF₃, —OCF₃, halogen, —NO₂, —CN, C₁-C₆alkyl, C₁-C₆alkoxy, —NH₂, (C₁-C₆)-alkyl-amino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —CONR²⁴R²⁵, —NHCOR¹⁶, —NHSO₂R¹⁶, —NHSO₂CH₂CF₃, —SO₂NR²⁴R²⁵, —NR²⁹C(O) NR²⁴R²⁵, —SO₂R³⁰, —P(O)(OR²⁹)₂, aryl, aryl(C₁-C₆)alkyl, heteroaryl, heterocycloalkyl, and —CR²⁹(═NOR²⁸);
R²² is —COR²³, —S(O)R³¹, —S(O)₂R³¹, —SO₂NR²⁴R²⁵ or —COOR²⁷;
R²³ is halo (C₁-C₆)alkyl; C₂-C₆alkenyl; halo (C₂-C₆)alkenyl; C₂-C₆alkynyl; C₃-C₇-cycloalkyl; (C₃-C₇)cycloalkyl(C₁-C₆)alkyl; (C₃-C₇)cycloalkyl substituted by 1 to 3 substituents selected from the group consisting of halo, (C₁-C₃)alkoxy(C₁-C₃)alkyl, hydroxy and C₁-C₆alkoxy; aryl; aryl(C₂-C₆)alkyl; heteroaryl; heterocycloalkyl; (C₁-C₆)alkyl substituted by 1-3 substituents independently selected from —COOH and —SO₃H; or 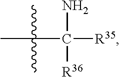
wherein R³⁵ and R³⁶ are independently selected from the group consisting of H, alkyl, or R³⁷-substituted C₁-C₆alkyl, wherein R³⁷ is selected from the group consisting of HO—, HS—, CH₂S—, —NH₂, phenyl, p-hydroxyphenyl and indolyl;
R²⁴ and R²⁵ are independently selected form the group consisting of H, C₁-C₆alkyl, halo(C₁-C₆)alkyl, C₂-C₆alkenyl, halo(C₂-C₆)alkyl, C₂-C₆alkynyl, aryl, aryl-(C₁-C₆)alkyl, C₃-C₇-cycloalkyl, halo(C₃-C₇)cycloalkyl, (C₁-C₃)alkoxy(C₁-C₃)-alkyl, hydroxy and C₁-C₆alkoxy;
R²⁶ is C₃-C₇-cycloalkyl, aryl, aryl-(C₁-C₆)alkyl, heteroaryl, heteroaryl-(C₁-C₆)alkyl or (C₁-C₆)alkylamino;
R²⁷ is C₁-C₆alkyl, phenyl, benzyl, (C₁-C₃)alkoxy(C₁-C₃)-alkyl, (C₃-C₇)-cycloalkyl, carboxy(C₁-C₆)alkyl, sulfo(C₁-C₆)alkyl, or (C₁-C₆)alkyl substituted by NR¹⁸R¹⁹ and carboxy;
R²⁸ is H, C₁-C₆alkyl, phenyl, benzyl or (C₁-C₆)alkoxy(C₁-C₃)alkyl;
R²⁹ and R³⁰ are independently selected from the group consisting of H and C₁-C₆alkyl;
R³¹ is (C₁-C₆)alkyl; halo(C₁-C₆)alkyl; C₂-C₆alkenyl; halo(C₂-C₆)alkyl; C₂-C₆alkynyl; C₃-C₇-cycloalkyl; (C₃-C₇)cycloalkyl substituted by 1 to 3 substituents selected from the group consisting of halo, (C₁-C₃)alkoxy(C₁-C₃)alkyl, hydroxy and C₁-C₆alkoxy; aryl; aryl (C₁-C₆)alkyl; heteroaryl; heterocycloalkyl; (C₁-C₆)alkyl substituted by 1-3 substituents independently selected from —COOH and —SO₃H; or (C₁-C₆)alkoxy;
R³² is R³⁵—(C₁-C₆)alkyl, R³⁵—(C₃-C₇)cycloalkyl, R³⁵—(C₂-C₆)alkenyl, R³⁵—(C₂-C₆)— alkynyl or R³⁵-aryl, wherein R³⁵ is 1 or 2 substituents independently selected from the group consisting of H, —COOH, —NH₂, —SO₃H, ═O and ═NOR²⁸; and
R³³ and R³⁴ are independently selected from the group consisting of H, (C₁-C₆)alkyl and C₃-C₇-cycloalkyl.

27. The method of claim 26, wherein the TG-2 inhibitor further inhibits the expression/activity of at least one of muscarinic receptor M₂ or muscarinic receptor M₃.

28. The method of claim 26, wherein the TG-2 inhibitor is administered to the subject in the range of about 0.010 μM to about 100 μM or in the range of about 0.010 μM to about 1 μM, or in the range of about 1 μM to about 10 μM.

29. The method of claim 26, wherein the subject is between 3 years old to 17 years old, or 4 years old to 14 years old.

30. The method of claim 26, wherein the subject is a mammal selected from a Human, Rodentia, Canis, Ungulate, Felidae, Leporidae, and Macaque.

31. The method of claim 26, wherein the subject is a human.

32. The method of claim 26, wherein the nucleic acid molecule is an RNA, a DNA, an aptamer, a micro RNA (miRNA) molecule or a small interfering RNA (si-RNA) molecule.

33. The method of claim 26, wherein the TG-2 inhibitor is formulated into a pharmaceutical composition further comprising a pharmaceutically acceptable carrier or excipient.

34. The method of claim 33, wherein the pharmaceutically acceptable carrier or excipient is selected from cellulose, hydroxymethylcellulose, cellulose acetate phthalate (CAP), gellan gum, polyalcohol, polyvinyl alcohol, hyaluronic acid, polyacrylic acid, carbopol polymer, poloxamer, poly(oxyethylene) and poly(oxypropylene) and block copolymers thereof, polyethylene oxide, polycarbophil, chitosan, cyclodextrin, liposome, nanoparticle, microparticle including microsphere and nanosphere, an ocular insert, ocular disc, soft contact lens, niosome, pharmacosome, collagen shield, ocular film and combinations thereof.

35. The method of claim 33, wherein the pharmaceutical composition is administered into the subject via topical, intra-ocular or systemic administration.

36. The method of claim 26, wherein myopia is selected from axial myopia, refractive myopia, curvature myopia, index myopia, degenerative myopia, nocturnal myopia, pseudomyopia, induced myopia, form deprivation myopia, congenital myopia, youth onset myopia, school myopia, early adult onset myopia and late adult onset myopia.

37. A method of identifying a TG-2 inhibitor comprising:
a) contacting a TGM-2 expressing cell with a solution containing a candidate TG-2 inhibitor; and
b) measuring inhibition of TGM-2 expression in the cell,
wherein the TG-2 inhibitor inhibits at least one of expression and activity of at least one of a muscarinic receptor M₂ and a muscarinic receptor M₃ in the cell, relative to the respective expression and activity measured in a control cell that has not been exposed to the candidate TG-2 inhibitor.

38. The method of claim 37, further comprising comparing (i) the TGM-2 expression of the cell that has been contacted with the candidate TG-2 inhibitor in step b) to (ii) TGM-2 expression of the control cell; and identifying therefrom the TG-2 inhibitor.

39. The method of claim 37, wherein the cell is derived from a mammal.

40. The method of claim 39, wherein the mammal is selected from a mammal that belongs to one of Rodentia, Canis, Ungulate, Felidae, Leporidae, and Macaque.

41. The method of claim 37, wherein the cell is derived from a human.

42. The method of claim 37, wherein the cell is selected from an epithelial cell, a subepithelial cell, a stromal cell, a cell membrane, an endothelial cell, a fibroblast cell and combinations thereof.

43. The method of claim 37, wherein the cell is derived from a tissue or a part thereof.

44. The method of claim 43, wherein the tissue is an eye tissue or a part thereof.

45. The method of claim 43, wherein the tissue or part thereof is selected from sclera, cornea, retina pigment epithelium (RPE), choroid, conjunctiva and combinations thereof.
